(19) United States
(12) Patent Application Publication
(10) Pub. No.: US 2011/0159524 A1
(43) Pub. Date: Jun. 30, 2011

(54) METHOD FOR MONITORING HYDROLYTIC ACTIVITY

(75) Inventors: Peter Helmuth Karuso, Killara (AU); Hung-Yoon Choi, Rydalmere (AU)

(73) Assignee: FLUOROTECHNICS PTY LIMITED, North Ryde (AU)

(21) Appl. No.: 12/953,063

(22) Filed: Nov. 23, 2010

Related U.S. Application Data
(63) Continuation of application No. 12/084,493, filed on May 2, 2008, now abandoned.

(30) Foreign Application Priority Data
Nov. 4, 2005 (AU) 2005906136

(51) Int. Cl. C12Q 1/25; C12Q 1/37; C12Q 1/44; C12Q 1/42; C12Q 1/34

(52) U.S. Cl. 435/7.72; 435/4; 435/23; 435/19; 435/21; 435/18; 435/24

(57) ABSTRACT

The present invention relates to methods of measuring the activity of a hydrolytic agent comprising contacting a biomolecule with a hydrolytic agent in the presence of a fluorescent dye under conditions that allow digestion of the biomolecule by the hydrolytic agent. The fluorescence of the dye is monitored over time and a change in fluorescence signifies digestion of the biomolecule by the hydrolytic agent. The biomolecule is preferably a protein, peptide or proteome but can also be a carbohydrate, oligonucleotide or lipid. Further methods relate to determining an end point for digestion of a biomolecule by a hydrolytic agent, and methods of monitoring digestion of a biomolecule by a hydrolytic agent. The monitoring can be performed on the reaction mixture in real time or via sampling. The invention also relates to kits for carrying out the method.

### TECHNICAL FIELD

The present invention relates to methods of monitoring the activity of a hydrolytic enzyme and to methods of monitoring hydrolytic digestion of biological macromolecules. In particular, the present invention is concerned with methods of monitoring protein and/or peptide digestion, or protease activity, using fluorescent dyes.

### BACKGROUND

Hydrolases are essential to many biological processes including apoptosis, cell differentiation, bone remodelling, blood clotting, disease states, cancer invasion, cell signalling and the infective cycle of many pathogenic organisms to name a few. The large range of known hydrolases with varying substrate specificity has led to the development of a multitude of assays that cannot be readily compared to each other because of the need for different, specialised substrates.

Generally, for example, for protease enzymes, these assays rely on peptide analogues of protease substrates monitored continuously through spectrophotometric changes (absorption or fluorescence) that occur after hydrolytic bond cleavage (eg WO 2003/089663 and references therein). This method is useful for exploring primary sequence specificity, measuring the activity of a specific hydrolase and for analysis of putative inhibitors but cannot be used to compare the activity of different proteases because there are limited substrate choices and those that are available are suitable for only a few proteases. Alternatively, a general substrate protein such as casein or BSA can be heavily labelled with a fluorophore and the decrease in quenching used as a measure of protease activity (e.g. Jones, et al. Analytical Biochemistry (1997) 251(2), 144-152). However, the substrate is heterogeneously labelled and the resulting peptides variably labelled, not allowing subsequent peptide analysis for the exploration of sequence specificity. Physically labelling a protein will also affect the ability of proteases to digest the protein.

Spencer et al. (Anal. Biochem. (1975) 64, 556-566) have reported a method to follow hydrolytic activity of some proteases on intact protein substrates that contain hydrophobic pockets, such as BSA, α-casein, urease and catalase, by the release of the fluorophore, 1-anilino-8-naphthalenesulfonate from these pockets. However, the method is not generic; working only for proteins that contain a hydrophobic pocket.

The prior art contains methods that require dye-labelled protease substrates. However, these methods perturb the substrate because the labels are invariably non-proteinaceous in nature and often very bulky, raising doubts about their utility as reasonable models for the proteases' natural substrate(s). Dye free methods include electrophoresis, HPLC and mass spectrometry. These methods are, however, laborious, not suitable for real-time measurements, and kinetic data are difficult to extract.

Further, proteolytic digestion with a range of proteases is a commonly used as a first and important step in techniques for protein identification in proteomics.

The hydrolysis of DNA has been measured by displacement of DNA-binding dyes such as PicoGreen (Tolun, et al., Nucleic Acids Research (2003) 31(18), e111/1-e111/6) or ethidium bromide (e.g. Ferrari et al., Nucleic Acids Research (2002) 30(20), e112/1-e112/9). These methods do not rely on fluorescently labelled substrate but on the uniform structure of DNA to allow real-time monitoring of nuclease or helicase activity by displacement of the dye from the DNA minor groove or from intercalation sites. It would be advantageous if this type of assay could be developed for other hydrolases such as proteases, esterases, glycosylases, phosphatases etc.

Consequently there is a high demand for quick and easy real-time assays for hydrolase activity that are quantitative and allow the comparison of, for example, different proteases and which are substrate independent. Even more demanding is proteomics, where there is the need for measuring hydrolase activity in a way that allows the fragments generated to be analysed by mass spectrometry or HPLC. Thus, there also exists a need for new and versatile approaches to monitoring the progress of protein or peptide digestion where the peptides and other fragments generated are available for further down-stream analysis.

It is therefore an object of the present invention to overcome or ameliorate at least one of the disadvantages of the prior art, or to provide a useful alternative.

### SUMMARY OF THE INVENTION

According to a first aspect the invention provides a method of measuring the activity of a hydrolytic agent comprising:
step 1: contacting a biomolecule with a hydrolytic agent in the presence of a fluorescent dye under conditions which allow digestion of the biomolecule by the hydrolytic agent; and
step 2: monitoring fluorescence of the dye over time, wherein a change in fluorescence over time signifies digestion of the biomolecule by the hydrolytic agent.

The biomolecule may be any biological macromolecule. However, the biological macromolecule is preferably a protein, peptide or proteome. The change may be an increase or a decrease in fluorescence.

According to a second aspect the invention provides a method of monitoring digestion of a biomolecule by a hydrolytic agent comprising:
step 1: contacting a biomolecule with a hydrolytic agent in the presence of a fluorescent dye under conditions which allow digestion of the biomolecule by the hydrolytic agent, and
step 2: monitoring fluorescence of the dye over time, wherein a change in fluorescence over time signifies digestion of the biomolecule by the hydrolytic agent.

According to a third aspect the invention provides a method of determining an end-point for digestion of a biomolecule by a hydrolytic agent comprising:
step 1: contacting a biomolecule with a hydrolytic agent in the presence of a fluorescent dye under conditions which allow digestion of the biomolecule by the hydrolytic agent, and
step 2: monitoring a change in fluorescence of the dye over time, wherein the absence of a further change in fluorescence signifies the end-point for digestion of the biomolecule.

According to a fourth aspect the invention provides a method of monitoring digestion of a biomolecule by a hydrolytic agent comprising:
step 1: contacting a biomolecule with a hydrolytic agent to form a reaction mixture,
step 2: contacting a first sample of the reaction mixture with a fluorescent dye and determining fluorescence of first sample,
step 3: subjecting the reaction mixture of step 1 to conditions which allow digestion of the biomolecule by the hydrolytic agent, and
step 4: at a desired time point during digestion of the biomolecule, contacting a second sample of the reaction mixture with a fluorescent dye; and
step 5: determining fluorescence of the second sample, wherein a change in fluorescence of the second sample when compared to the first sample signifies the degree of digestion of the biomolecule by the hydrolytic agent.

An embodiment of the above method contemplates sampling the reaction mixture at regular intervals during digestion and, after addition of a fluorescent dye to each of the samples, measuring a change in fluorescence over time until no further decrease in fluorescence is observed. This variant of the method can be used to determine the end point of digestion of a biological macromolecule. Advantageously, fluorescence is measured over time to provide data indicative of a reaction rate coefficient. The sub-sampled reaction mixtures are suitably quenched prior to measurement.

According to a fifth aspect the present invention provides a fluorescent dye or a composition thereof for use in the methods of any one of previous aspects.

According to a sixth aspect the present invention provides a kit for use in the method of any one of the previous claims comprising: a fluorescent dye, one or more hydrolytic agents, optionally a standard substrate for the hydrolytic agent, and instructions on how to use the kit for monitoring digestion of the biological macromolecule.

Preferably the kit includes a standard protein or peptide substrate or any other biological standard.

Preferably the kit includes standard buffers appropriate for the enzyme. The preferred buffer comprises one of the Good's buffers such as bicine, BES etc.

Any hydrolysable biomolecule may be used in the present invention.

The biomolecule may be of any size/molecular weight but is preferably a macromolecule. Most preferably the macromolecule is a carbohydrate, lipid, peptide/protein, proteome, phosphoprotein, glycoprotein or oligonucleotide.

It will be clear to the skilled addressee that in the context of the present invention, the term “biomolecule” includes both naturally occurring molecules and synthetic molecules wherein the synthetic molecules may include moieties similar to those found in naturally occurring molecules; or analogues, homologues, derivatives or modifications thereof (wherein the modifications may be made either by/within an organism or by synthetic means.)

Typically, the biomolecules of the invention are oligomers/polymers of amino acids formed by two or more amino acids i.e. peptides, polypeptides or proteins of any size; oligomers/polymers formed by two or more nucleic acids eg. DNA (including cDNA, gDNA and any non-coding DNA) or RNA (including mRNA, tRNA, RNAi, siRNA or any non-coding RNA, etc); or oligomers/polymers found in lipids or parts thereof.

It would be clear to the skilled person that the present invention also relates to a mixture of biomolecules.

Any hydrolysable biological macromolecule may be used. However, the biological macromolecule is preferably a carbohydrate, oligonucleotide, protein, peptide, lipid or mixtures thereof. The biomolecule may be present in a genome, proteome or cellular extract.

Alternatively, preferably the biological macromolecule is a protein, a peptide or proteome capable of being cleaved or digested by a hydrolytic agent.

Preferably the substrate has enhanced hydrophobicity. Any means that provides such enhanced hydrophobicity would be suitable. However, a protein denaturant, which in preferred embodiments is a detergent is used in non-denaturing amounts to enhance protein hydrophobicity, thereby enhancing or changing binding of the fluorescent dye. Such detergents include but are no limited to SDS, LDS, triton X-100, CHAPS, ALS, CTAB, DDAO, DOC, etc.

Preferably the hydrolytic agent changes the hydrophobicity of the biomolecule.

Throughout this specification the term protein and proteins are to be taken to include, inter alia, recombinant protein(s). The protein or peptides may be present in a complex protein/peptide mixture, for example an entire proteome.

Preferably the hydrolytic agent is an enzyme and even more preferably it is a proteolytic agent such as a protease, esterase, glycosylase, phosphatase or nuclease capable of cleaving a biomolecule in at least one position. Non-limiting examples of hydrolases that can be used in the present invention are carboxylic ester hydrolases, thiolester hydrolases, phosphoric monoester hydrolases, phosphoric diester hydrolases, triphosphoric monoester hydrolases, sulfuric ester hydrolases, diphosphoric monoester hydrolases, phosphoric triester hydrolases, exodeoxyribonucleases producing 5′-phosphomonoesters, exoribonucleases producing 5′-phosphomonoesters, exoribonucleases producing 3′-phosphomonoesters, exonucleases active with either ribo- or deoxyribonucleic acid, exonucleases active with either ribo- or deoxyribonucleic acid, endodeoxyribonucleases producing 5′-phosphomonoesters, endodeoxyribonucleases producing other than 5′-phosphomonoesters, site-specific endodeoxyribonucleases specific for altered bases, endoribonucleases producing 5′-phosphomonoesters, endoribonucleases producing other than 5′-phosphomonoesters, endoribonucleases active with either ribo- or deoxyribonucleic, endoribonucleases active with either ribo- or deoxyribonucleic acids, glycosidases (i.e. enzymes hydrolyzing O- and S-glycosyl), enzymes hydrolyzing N-glycosyl compounds, thioether and trialkylsulfonium hydrolases, ether hydrolases, aminopeptidases, dipeptidases, dipeptidyl-peptidases and tripeptidyl-peptidases, peptidyl-dipeptidases, serine-type carboxypeptidases, metallocarboxypeptidases, cysteine-type carboxypeptidases, omega peptidases, serine endopeptidases, cysteine endopeptidases, aspartic endopeptidases, metalloendopeptidases, threonine endopeptidases.

The preferred fluorescent dyes are those that bind or interact with proteins or peptides hydrophobicly. In one embodiment the fluorescent dye is SYTOX green. In another embodiment, the fluorescent dye is Hoechst 33342. In another embodiment, the fluorescent dye is propidium iodide. In another embodiment, the fluorescent dye is ANS. In another embodiment, the fluorescent dye is epicocconone. In another embodiment, the fluorescent dye is Nile red. In another embodiment, the fluorescent dye is BODIPY FL C₅ ceramide. In another embodiment, the fluorescent dye is 5-octadecanoylaminofluorescein. In another embodiment, the fluorescent dye is SYPROorange. In another embodiment, the fluorescent dye is a cyanine dye. In another embodiment, the fluorescent dye is chosen from the laurdan/prodan family of dyes. In another embodiment, the fluorescent dye is a dapoxyl derivatives. In another embodiment, the fluorescent dye is a pyrene dye. In another embodiment, the fluorescent dye is a diphenylhexatriene derivative. In another embodiment, the fluorescent dye is a rhodamine derivative. In another embodiment, the fluorescent dye is a coumarin derivative. However, it will be clear from the teaching herein that any dye which is hydrophobicly active will be useful in the methods of the present invention. Examples of useful dyes are the cyanine dyes, laurdan/prodan family of dyes, dapoxyl derivatives, pyrene dyes, diphenylhexatriene derivatives ANS and its analogues, styryl dyes, Nile red, amphiphilic fluorescein, rhodamines and coumarins or any other fluorophore that substantially changes its fluorescent behaviour in response to the lipophilicity of its environment (hydrophobicly active). Further, it is clear from the teachings herein that enzymes that derivatise biomolecules such as transferases (eg methyltransferases, hydroxymethyl-, formyl- and related transferases, carboxyl- and carbamoyltransferases, amidinotransferases, transketolases and transaldolases, acyltransferases, glycosyltransferases, hexosyltransferases, pentosyltransferases, enzymes transferring other glycosyl groups, enzymes transferring alkyl or aryl groups, transaminases (aminotransferases), oximinotransferases, enzymes transferring phosphorous-containing groups such as protein kinases, sulfurtransferases, sulfotransferases, CoA-transferases and selenotransferases) that change the hydrophobicity of the protein would interact with the mentioned dyes to allow real-time monitoring of transferase activity.

Preferably the fluorescent dye substantially changes its fluorescent behaviour in response to the lipophilicity of its environment. Preferably hydrolysis of the biomolecule is substantially unaffected by the fluorescent dye.

In the context of the present invention the term “epicocconone and related dyes” is intended to encompass epicocconone itself as well as related fluorescent dyes as specifically disclosed in WO 2004/085546 incorporated in its entirety herein by reference.

According to a seventh aspect the present invention provides a method for measuring and/or detecting products of a hydrolytic digestion reaction comprising:
step 1: subjecting a biomolecule to hydrolytic digestion to obtain protein or peptide fragments,
step 2: contacting said protein or peptide fragments with a fluorescent dye, and
step 3: detecting a change in fluorescence of the dye, wherein said change in fluorescence of the dye is proportional to the quantity of said protein or peptide fragments.

A method according to any one of the preceding claims wherein said biomolecule is a biological macromolecule.

Preferably the hydrolysis is carried out in the presence of a buffer, such as a Good's buffer or a bicine buffer.

Preferably fluorescence is measured over time to provide data indicative of a reaction rate coefficient. Preferably the digestion is stopped when an end point is achieved and further analysis of the reaction mixture takes place after digestion is stopped. The further analysis may be selected from the group consisting of peptide mass finger printing (PMF), peptide mapping and HPLC.

In other embodiments a base may be added to the fluorescent dye.

In further embodiments the biomolecule is derived from a biological sample or food sample. The biomolecule may be a protein or mixture of proteins. Preferably the biomolecule is a carbohydrate or mixture of carbohydrates. Preferably the biomolecule is a glycoprotein or starch. Preferably the said biomolecule is a lipid. Preferably the biomolecule is a vegetable oil. Preferably the biomolecule is an oligonucleotide. Preferably the biomolecule is DNA.

The kit may also include a standard protein or peptide substrate chosen from the group consisting of BSA, apo-transferrin, α-casein, β-casein, carbonic anhydrase, fetuin, salmon sperm DNA, soluble starch, and olive oil.

### BRIEF DESCRIPTION OF THE FIGURES

Preferred embodiments of the invention will now be described, by way of example only, with reference to the accompanying drawings in which:

FIG. 1. Kinetics of real-time monitoring of PNGase F deglycosylation of fetuin using epicocconone as a reporter dye (A; λₑₓ 540±10 nm, λₑₘ 630±10 nm), and validation of the digests by SDS-PAGE (B). The glycoprotein alone with fluorophore (open squares) is fitted to a two-phase exponential association/dissociation model (Y=Ymax*exp(1−k1X)+span*exp(−k2X)+plateau) and the determined value for k1 and k2 are used as the fixed values for k1 and k2 in a three phase exponential association/dissociation equation (Y=span1*exp(1−k1X)+span2*exp(−k2X)+span3*exp(1−k3X)+plateau) for the enzymic hydrolysis of fetuin with the enzyme PNGase F(open circles). The inset shows the derived kinetic constants and half-life for hydrolysis. B represents SDS-PAGE validation of native (lane 2) and deglycosylated fetuin (lane 3): Lane 1 represents LMW marker (97, 66, 45, 30, 20.1, and 14.4 kDa) and lane 4 is just the enzyme PNGase F (48.4 kDa).

FIG. 2. Kinetics of real-time monitoring of hydrolysis of salmon sperm double stranded DNA by DNase 1 using Hoechst 33342 (λₑₓ 355 nm, λₑₘ 460 nm) (A), SYTOX-green (λₑₓ 485 nm, λₑₘ 520 nm (B) and propidium iodide (λₑₓ 540±10 nm, λₑₘ 630±10 nm) (C) as reporter dyes. Progress curves are fitted to fluorescence data for DNA with dye (open squares) and DNA with DNase and dye (open circles). In each case, the protein with fluorophore is fitted to a single phase exponential decay (Y=span*exp(−kX)+plateau) and the value for k used as the fixed value for k1 in a two phase exponential decay (Y=span1*exp(−k1X)+span2*exp(−k2X)+plateau) for the DNA plus DNase. The insets provide the pseudo-first order kinetic constants and half-lives of hydrolysis. D represents DNA gel electrophoresis-based validation of hydrolysis of DNA samples using Hoechst 33342 (lane 2 and 3), SYTOX-green (lane 4 and 5) and propidium iodide (lane 6 and 7): Lane 1 and 8 represent SPP1 DNA molecular weight markers.

FIG. 3. Kinetics of real-time monitoring of hydrolysis of starch by α-amylase using epicocconone (λₑₓ 540±10 nm, λₑₘ 630±10 nm) as a reporter dye. A is amylase with starch followed by a decrease in fluorescence of epicocconone and B is with triton X-100 (0.02%) detergent added. In each case, the protein with fluorophore (open squares) is fitted to a single phase exponential decay (Y=span*exp(−kX)+plateau) and the value for k used as the fixed value for k1 in a two phase exponential decay (Y=span1*exp(−k1X)+span2*exp(−k2X)+plateau) for starch plus amylase (open circles). The insets provide the pseudo-first order kinetic constants and half-lives of hydrolysis.

FIG. 4. Kinetics of real-time monitoring of dephosphorylation of β-casein (β-CN) by an alkaline phosphatase using BODIPY® FL C₅-ceramide (λₑₓ 485 nm, λₑₘ 520 nm) as a reporter dye. The phosphoprotein alone with fluorophore (open squares) is fitted to a one-phase exponential growth (Y=span*exp(1−kX)+plateau) and the determined value for k used as the fixed value for k1 in a two phase exponential growth (Y=span1*exp(1−k1X)+span2*exp(1−k2X)+plateau) for the enzymic hydrolysis of the phosphoprotein with phosphatase (open circles). The inset shows the derived kinetic constants and half-life for hydrolysis of β-casein with alkaline phosphatase.

FIG. 5. Kinetics of real-time monitoring of hydrolysis of olive oil by lipase (0.01 μL, Greasex®) using 5-octadecanoylaminofluorescein (λₑₓ 485 nm, λₑₘ 520 nm) as a reporter dye. The squares are the real-time data of olive oil with no enzyme and the circles are olive oil with lipase added. The olive oil alone with fluorophore (open squares) is fitted to a one-phase exponential decay (Y=span*exp(−kX)+plateau) and the determined value for k used as the fixed value for k1 in a two phase exponential decay (Y=span1*exp(−k1X)+span2*exp(−k2X)+plateau) for the enzymic hydrolysis of the oil with a lipase (open circles). The inset shows the derived kinetic constants and half-life for hydrolysis.

FIG. 6. Example of real-time monitoring of a non-specific protease (papain) digestion of four different proteins followed with the fluorophore epicocconone (λₑₓ 540±10 nm, λₑₘ 630±10 nm) as a reporter dye. In each case, the protein with fluorophore (open squares) is fitted to a single phase exponential decay (Y=span*exp(−kX)+plateau) and the value for k used as the fixed value for k1 in a two phase exponential decay (Y=span1*exp(−k1X)+span2*exp(−k2X)+plateau) for the protein plus papain (open triangles). BSA (A), casein (B), apo-transferrin (C) and carbonic anhydrase (D) are shown as examples. The insets provide the pseudo-first order kinetic constants and half-lives of digestion. E represents SDS-PAGE validation of different proteins digested with papain: Lane 1, LMW marker (97, 66, 45, 30, 20.1, and 14.4 kDa); 2, BSA; 3, BSA/papain; 4, α-casein; 5, casein/papain; 6, apo-transferrin; 7, apo-transferrin/papain; 8, carbonic anhydrase (bovine); 9, carbonic anhydrase/papain; 10, papain only.

FIG. 7. This example shows the application of a variety of fluorophores for the monitoring of BSA hydrolysis by proteases. Real-time monitoring of proteolysis with trypsin using SYPROorange (A), Nile red (B) and epicocconone (C), and with papain using ANS (D). In each case, the protein alone with fluorophore (open squares) is fitted to a one-phase exponential decay (Y=span*exp(−kX)+plateau) and the determined value for k used as the fixed value for k1 in a two phase exponential decay (Y=span1*exp(−k1X)+span2*exp(−k2X)+plateau) for the protein plus protease (open squares). The insets in each graph show the derived kinetic constants and half-life for hydrolysis. E represents SDS-PAGE validation of BSA proteolysis with trypsin (lanes 2-7) and papain (lanes 8-9) using different fluorphores: lane 2 and 3, SYPROorange; lane 4 and 5, Nile red; lane 6 and 7, epicocconone; lane 8 and 9, ANS. Lane 1 represent a LMW marker (97, 66, 45, 30, 20.1, and 14.4 kDa from top to bottom).

FIG. 8. Tryptic digestion of bovine serum albumin (BSA; open circles) and carbonic anhydrase (CA; inverted triangles) was carried out without the inclusion of a reporter dye. Each reaction was sub-sampled at the indicated time points and the trypsin activity quenched with either leupeptin (A) or soybean trypsin inhibitor (B) before adding a reporter dye (epicocconone) and reading of fluorescence. The inset shows the apparent first order rate constant of digestion calculated by fitting the data to a single-phase exponential decay

FIG. 9. Kinetics of real-time monitoring of trypsin digestion of a complex proteome (yeast) with a hydrolytic enzyme (trypsin) using a fluorescent reporter dye (epicocconone; λₑₓ 540±10 nm, λₑₘ 630±10 nm). The glycoprotein alone with fluorophore (open squares) is fitted to a two-phase exponential association/dissociation model (Y=Ymax*exp(1−k1X)+span*exp(−k2X)+plateau) and the determined value for k1 and k2 are used as the fixed values for k1 and k2 in a three phase exponential association/dissociation equation (Y=span1*exp(1−k1X)+span2*exp(−k2X)+span3*exp(1−k3X)+plateau) for the enzymic hydrolysis of the yeast proteome with the enzyme trypsin F(open circles). The inset shows the derived kinetic constants and half-life for hydrolysis. The goodness of fit is shown by the residuals for the proteome plus dye (solid squares) and the proteome plus hydrolase and dye (solid circles).

FIG. 10. Real-time monitoring of tryptic digestion of carbonic anhydase (CA) with and without inclusion of a non-denaturing quantity of a detergent (SDS) with epicocconone as the reporter dye. CA plus SDS but with no trypsin (open squares) shows a slow decline in fluorescence whereas in the presence of trypsin (open circles) there is an exponential decay in fluorescence. Without the presence of SDS the signal is much lower (inverted triangles) due to the decreased hydrophobicity around the CA.

FIG. 11. Raw data for the change of fluorescence of bicine buffer (blue), trypsin (yellow), Bovine serum albumin (magenta) and a tryptic digest of BSA (cyan).

FIG. 12. Gel electrophoresis of subsampled BSA tryptic digest (containing epicocconone) from 0-128 minutes, quenched in gel loading buffer at 85° C. (A). A control gel, containing all components except trypsin (B) shows no change over 128 minutes. Gels were stained with Deep Purple Total Protein Stain. The first lane showed a LMW marker and the last lane overnight incubation.

FIG. 13. Total fluorescent intensity measured from gated regions of the sub-samples shown in FIG. 12.

FIG. 14. Kinetic analysis of the raw data from FIG. 11. A is the analysis of the reaction of epicoccone in bicine buffer (pH 8.4) showing development of the stain and subsequent decomposition. Apparent first order constants are indicated. The rate of decomposition was used in calculating the first order rate constant for the tryptic digest (B). A similar result was obtained by analysis of the data from protein gels (C) from FIG. 13

FIG. 15. Visualization of trypsin digests in SDS-PAGE. Lanes 1: Marker; 2: trypsin only (T=0); 3: T=0 (no trypsin); 4: T=0 (immediately after trypsin added); 5: T=0.25 h; 6: T=0.5 h; 7: T=1 h; 8: T=6 h; 9: T=18 h; 10: trypsin only (T=18)

FIG. 16. Real-time monitoring of chymotrypsin kinetics in the digestion of BSA followed by epicocconone.

FIG. 17. Real-time monitoring of trypsin kinetics in the digestion of BSA followed with epicocconone.

FIG. 18. Real-time monitoring of trypsin kinetics in the digestion of BSA followed by SYPROorange. The upper graph shows the response of SYPROorange to BSA over time (r²=0.9995) and the lower graph shows the same except with the addition of trypsin. The rate of trypsin hydrolysis was determined to be 0.1466 min⁻¹ (r²=0.9739).

FIG. 19. FluoroProfile assay. The 1^{st} and 2^{nd} raw were the duplicate samples of undigested BSA sample (no trypsin) incubated for 18 hour at 37° C. The 3^{rd} and 4^{th} row were the duplicate samples of digested BSA sample (trypsin) incubated for 18 hour at 37° C. The samples were serially diluted 4-fold to obtain 1 in 1024 dilution at the end (see captions). The 5^{th} and 6^{th} row were the BSA standard and aprotinin standard, respectively that were serially diluted from 250 μg mL⁻¹ to 61 ng mL⁻¹. Column 1 containing 50 mM bicine buffer as a control.

FIG. 20. Fluorescence was plotted against a 4-fold dilution series.

FIG. 21. Fluorescence vs. known BSA concentrations (The graph was plotted from Table A2).

FIG. 22. A. BSA standard curve. B. Aprotinin Standard Curve.

FIG. 23. Following tryptic digestion with Nile Red, another dye that increases fluorescence in hydrophobic environments. The upper graph show the response of Nile Red to BSA over time (r²=0.9969) and the lower graph the same except with the addition of trypsin. The rate of trypsin hydrolysis was determined to be 0.1302 min⁻¹ (r²=0.9894).

### DESCRIPTION OF THE PREFERRED EMBODIMENT

The present invention is based on a finding that a response of fluorescent dyes to a hydrophobic environment can be used to follow the activity of hydrolytic enzymes in a non-invasive way. As the dyes do not permanently covalently modify the substrate they do not significantly affect the activity of the enzymes. Without wishing to be bound by theory, the increase in hydrophilicity of the end product of hydrolysis results in a concomitant reduction in fluorescence by fluorophores that are sensitive to their environment.

More specifically, the present invention is based on a surprising finding that the fluorescence of a fluorescent dye, epicocconone, when used in a hydrolytic reaction comprising a protein and a hydrolytic enzyme (eg. papain or the like), decreases as the protein digestion progresses to completion.

Epicocconone, its derivatives and uses have been described in International Patent Application No. PCT/AU2004/000370 (PCT publication No. WO 2004/085546) incorporated in its entirety herein by reference. Epicocconone and related dyes have been used successfully inter alia for detection and quantification of proteins and other biological macromolecules. These methods are based on enhancement in the fluorescence of a dye such as epicocconone with increasing concentration of protein. With respect to the studies described herein it was hypothesized that fluorescence intensity of the digested protein samples would increase over time proportionally to an increase in exposure of lysine residues as a consequence of protein digestion. Unexpectedly, however, inclusion of epicocconone in a papain digest of BSA (bovine serum albumin) showed a rapid decrease in fluorescence, which effectively followed the digestion process and reached its lowest level at the completion of protein digestion, as confirmed by SDS-PAGE.

Without wishing to be bound by theory or any particular mechanism of action, it seems likely that the hydrolytic digestion of a protein or a peptide which results in smaller peptide fragments, alters the charge density or hydrophobicity of a protein thus affecting its interaction with a hydrophobicly active fluorescent dye, which in turn decreases the fluorescence of the dye. Further, the addition of a non-denaturing quantity of surfactant (eg SDS, triton X-100 or CTAB) dramatically increased the difference between the fluorescence of hydrolysed and unhydrolysed protein. This principle can be generally applied to all hydrolytic enzymes, or other hydrolytic agents, that release products that are more polar or less polar than the starting material and for all fluorophores that increase or decrease their quantum yields in response to the hydrophobicity of their environment.

As indicated above, it would be clear that the observed reduction in fluorescence of a hydrophobicly active dye such as epicocconone, during protein digestion by a protease would also apply to other hydrophobicly active fluorescent dyes. For example families of dyes such as the cyanine dyes, laurdan/prodan family of dyes, dapoxyl derivatives, pyrene dyes, diphenylhexatriene derivatives ANS and its analogues, styryl dyes, Nile red, amphiphilic fluorescein, rhodamines and coumarins or any other fluorophore that substantially changes its fluorescent behaviour in response to the lipophilicity of its environment. Other dyes with similar properties will be known to those skilled in the art.

Further, the above-mentioned principles, and effects observed with fluorescent dyes, should apply to any methods used for cleavage or digestion of proteins or the transfer of groups to a protein or biomolecules (eg transferases, kinases).

Based on the above principles, in one embodiment the invention relates to methods of measuring activity of a hydrolytic enzyme such as a protease, by combining the hydrolytic enzyme with a suitable substrate (eg. a protein or a peptide) and a fluorescent dye which is able to interact with the substrate, and measuring or observing the decrease or increase (change) in fluorescence over time, which is indicative of the activity of the hydrolytic enzyme. For such applications a standard protein substrate, for example BSA or similar, can be employed.

In another embodiment the invention relates to methods of monitoring the increase in fluorescence over time as polar groups such as phosphates, sulfates or carbohydrates are removed from a protein.

In another embodiment the invention relates to methods of monitoring, either in real time or by serial sampling, hydrolytic digestion of a biomolecule such as a protein in a reaction similar to that described above and again detecting or observing a decrease or increase in fluorescence over time as an indication of progress of hydrolytic digestion.

The methods described herein lend themselves easily to automation or continuous-flow techniques.

The invention will now be described in more detail, with reference to non-limiting examples. Examples of proteases provided herein include trypsin and papain, and of the fluorescent dyes include epicocconone, ANS, Nile red and SYPROorange, merely as convenient systems to demonstrate the principles and working of the invention. Other examples of hydrolytic enzymes provided herein include esterases (phosphatase, lipase, DNase) and glycosylases (amylase, PNGase) again merely for convenience to demonstrate the utility of the invention. Further examples of suitable fluorophores provided include SYTOX green, Hoechst 33342, propidium iodide, epicocconone, BODIPY FL C₅ ceramide or 5-octadecanoylaminofluorescein again merely for convenience to demonstrate the wide utility of the invention.

### PRELIMINARY EXAMPLES

### Example A

### Real-Time Monitoring of Trypsin Digestion Using Epicocconone

The aim of this investigate was to ascertain whether or not a fluorescent dye such as epicocconone can be used for real-time monitoring of tryptic protein digests.

### A.1 Materials

Bicine (50 mM, pH 8.4, Sigma-Aldrich B3876)
BSA (10 mg/mL in 50 mM Bicine, Sigma-Aldrich A3059)
Trypsin (20 μg/20 μL 1 mM HCl, Sigma-Aldrich T6567)
Idoacetamide (1 M in 100 mM bicine, Sigma-Aldrich I6125)
DTT (200 mM in 100 mM bicine, Bio-rad 161-0611)
96-well plate with clear bottom (Greiner bio-one, 655096)
Epicocconone (24 mM in DMSO, FLUOROtechnics)
Deep Purple total protein gel stain (GE Healthcare)
NuPAGE Novex 12% Bis-Tris Gels (Invitrogen, NP0341)
LMW Marker (Amersham Biosciences, 17-0446-01)

### A.2 Equipment

Typhoon 9200 (Amersham Biosciences)
FluoStar (BMG)
Electrophoresis system (XCell SureLock, Invitrogen)

### A.3 Methods

### A.3.1 Preparation of BSA for Digestion

1 Trypsin digestion was carried out in bicine buffer (ph 8.4).
2 BSA was prepared in 10 mg/mL in 50 mM bicine buffer.
3 One hundred microliter of the BSA sample was used for trypsin digestion.

### A.3.2 Reduction and Alkylation

1 The 100 μL of BSA sample was reduced by adding 5 μL of DTT stock for 10 min at 80° C.
2 The sample was alkylated by adding 4 μL of the iodoacetamide stock at room temperature for 45 min-1 hr.
3 The remaining iodoacetamide of the sample was neutralized by adding 20 μL of the DTT at room temp for 45 min-1 hr.

### A.3.3 Real-Time Monitoring of Trypsin Digestion Using Epicocconone (Fluostar Assay)

1 The reduced and denatured BSA sample from 3.2 above was diluted 10-fold in 50 mM bicine buffer (25 μL+225 μL bicine buffer). BSA molar concentration was calculated to be approx. 4 μM.
2 One hundred microliter of the sample (step 1) was prepared in duplicates and added to a microtiter plate. Controls included a bicine-based digestion buffer, a trypsin sample only and an undigested BSA sample (no trypsin).
3 Epicocconone stock solution was diluted 100-fold in 50 mM bicine. One hundred microliter of diluted epicocconone solution was added to each corresponding well. The final concentration was 12 μM. At this point in time, it required approximately 10 min to get appropriate FluoStar setting conditions.
4 Trypsin (Sigma-Aldrich T6567), reconstituted in 1 mM HCl, was added at a ratio of 1:40.

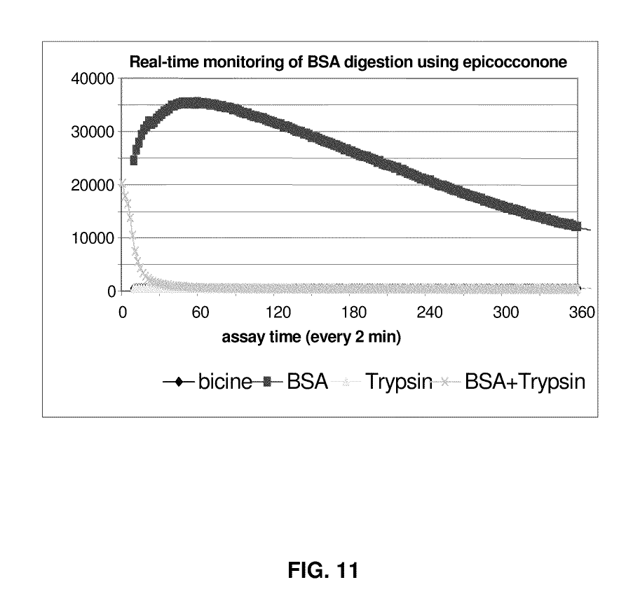

Fluorescence development was monitored in real time every 2 minutes up to 6 hours using FluoStar (Ex/Em=540/630±12). FluoStar settings were as follows: temperature, 37° C.; 10 flashes/cycle to 180 cycles.
6 The data were plotted in an Excel graph (FIG. 11).

### A.3.4 Visualization of Trypsin Digests in SDS-PAGE

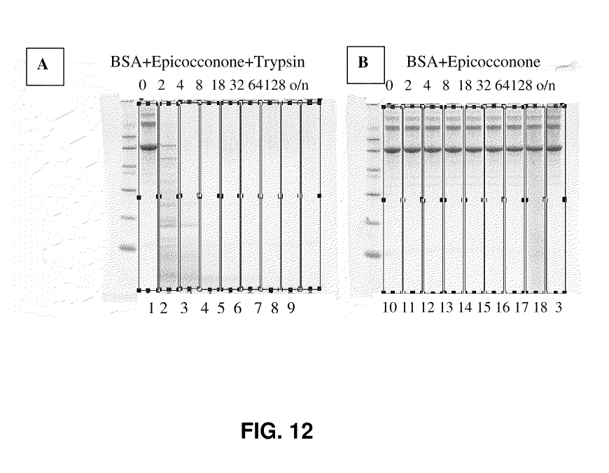

1 The reduced and denatured BSA sample from 3.2 above was diluted 10-fold in 50 mM bicine buffer (25 μL+225 μL bicine buffer). BSA molar concentration was calculated to be approx. 4 μM.
2 One hundred microliter of the sample (step 1) was added to a 1.5 mL microtube. Controls included a bicine-based digestion buffer and an undigested BSA sample (no trypsin).
3 Epicocconone stock solution was diluted 100-fold in 50 mM bicine. One hundred microliter of diluted epicocconone solution was added to each corresponding well. The final concentration was 12 μM. At this point (time 0), 15 μL of the samples were taken out, mixed with 15 μL of a protein gel loading buffer (2×), and denatured for 5 min at 85° C.
4 Trypsin, reconstituted in 1 mM HCl, was added at a ratio of 1:40. The sample tubes were then incubated at 37° C.
5 The sub-samples were collected 2, 4, 8, 16, 32, 64, 128 min, and overnight (18 hours). At each sampling point, 15 μL of the samples were taken out, immediately mixed with 15 μL of a protein gel loading buffer (2×), denatured for 5 min at 85° C., and stored at −80° C.
6 The sub-samples collected as described above were run by SDS-PAGE (Nupage, 12% BT gel), and gels were stained with Deep Purple for visualization.
7 The Deep Purple-stained gels were imaged by Typhoon scanner (Ex:Em=532:560 LP; 440 PMT).
8 All the gel lanes showing digested (FIG. 12A) and undigested (FIG. 12B) protein samples were equally gated, and the fluorescent intensity was measured by ImageQuant (v 5.2) software.

### A.4 Data Analysis and Results

The results of these studies are summarized in FIGS. 11 to 14.

The raw data obtained from the reaction of BSA and BSA+trypsin with epicocconone was measured as described and fitted to simple first order kinetic models using Prism (Version 4.0.3, GraphPad Software, San Diego Calif., USA).

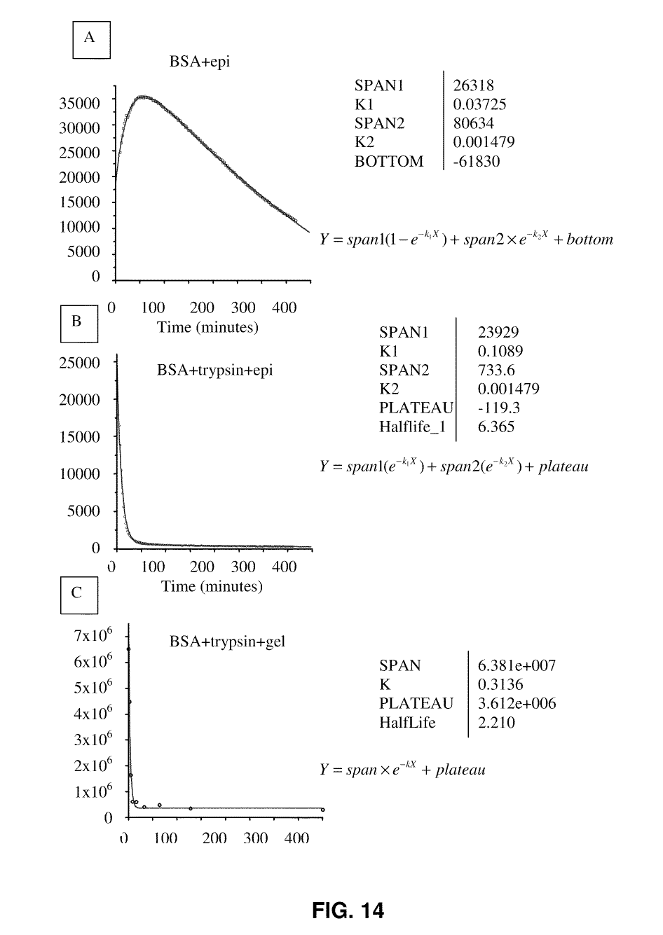

For the reaction of BSA with epicocconone in bicine buffer it is clear that there are at least two reactions, one is a time dependant association of epicocconone with BSA and then a slower decomposition, and/or photobleaching of this fluorescent conjugate. This process was modeled with a simple association/dissociation model Y=span1(1−e^{-k}^{₁}^{X})+span2×e^{-k}^{₂}^{X}+bottom where the first term refers to the exponential increase in signal and the second the exponential decrease. The values from FIG. 11 were subtracted from background fluorescence (bicine+trypsin) and adjusted for the actual start time of the experiment, estimated as 10 minutes from the first reading. An r² value of 0.9992 was obtained for this model (FIG. 14).

As the degradation of signal with time is a characteristic of the interaction of epicocconone with protein (and presumably peptides) the value for k₂ was used in the analysis of the trypsin kinetics. A two phase exponential decay (Y=span1(e^{k}^{₁}^{X})+span 2(e^{-k}^{₂}^{X})+plateau) was used were k₂ was set to the value found for BSA alone. Thus the estimated apparent first order rate constant for trypsin under the experimental conditions was found to be 0.109 min⁻¹ (r²=0.9871). Clearly the actual kinetics is more complex but this is a good approximation that would be of great utility for the monitoring of hydrolytic activity in many situations.

The results from the subsampled reaction was similarly analysed but without the term associated with degradation of the signal with time as this is not relevant in gels. Thus the apparent first order rate constant was found to be 0.3136 min⁻¹ (r²=0.9757). However, with so few points a large 95% confidence interval was found (0.1976 to 0.4296 min⁻¹). Coupled with the relatively imprecise mode of measurement (for example tryptic digestions continues during the subsampling procedure resulting in a higher apparent rate constant) suggests that in situ monitoring of tryptic digestion with epicocconone is comparable to the laborious process of subsampling and that the presence of the dye does not significantly affect the enzyme kinetics.

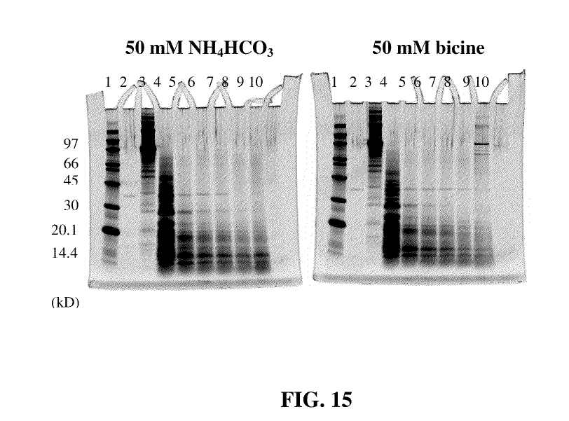

The experiment described above was also conducted in a carbonate buffer (NH₄HCO₃, 100 mM or 50 mM, pH 8.2) and results compared with data obtained using bicine buffer. The comparative results are shown in FIG. 15.

Sigma-Aldrich Proteomics grade trypsin (T 6567) worked well in both digestion buffers. The digestion appeared to reach completion within 0.5-1 hr.

The data presented indicate that reduced and alkylated BSA is rapidly digested with trypsin and that this process can be followed in situ with a fluorescent dye that is sensitive to its environment.

### Example B

### Kinetics of Chymotrypsin in BSA-Digestion Using Epicocconone

### B.1 Materials

Bicine (50 mM, pH 7.8, B3876)
BSA (10 mg/mL in 50 mM Bicine, A3059)
Chymotrypsin (C4129, 1 mg/mL of 1 mM HCl)
CaCl₂ (1M in RO water)
Idoacetamide (1 M in 100 mM bicine, 16125)
DTT (200 mM in 100 mM bicine, Bio-rad_161-0611)
Epicocconone (24 mM in DMSO, Fluorotechnics)
FluoStar (BMG)
96-well plate with clear bottom (Greiner bio-one, 655096)

### B.2. Methods

### B.2.1 Preparation of BSA for Digestion

1. Chymotrypsin digestion was carried out in bicine buffer.
2. BSA was prepared in 10 mg/mL in 50 mM bicine buffer.
3. One hundred microliter of the BSA sample was used for chymotrypsin digestion.

### B.2.2 Reduction, Aklylation and Neutralization

1. The BSA sample (100 μL) was reduced by adding 5 μL of DTT stock and heating (80° C.) for 10 min.
2. The sample was alkylated by adding the iodoacetamide (4 μL) stock at room temperature for 45 min-1 hr.
3. The remaining iodoacetamide of the sample was neutralized by adding DTT (20 μL) and incubating room temp for 45 min-1 hr.

### B.2.3 Real-Time Monitoring of Chymotrypsin Digestion Using Epicocconone (Fluostar Assay)

1. The reduced and denatured BSA sample from 2.2 was diluted 10-fold in 50 mM bicine buffer (25 μL+225 μL bicine buffer). BSA molar concentration was calculated to be approx. 6 μM.
2. One hundred microliter of the sample (step 1) was prepared in duplicates and added to a microtiter plate. Controls included a bicine-based digestion buffer, a chymotrypsin sample only and an undigested BSA sample (no chymotrypsin).
3. Epicocconone stock solution was diluted 100-fold in 50 mM bicine and 100 μL added to each well. The final concentration epicocconone was 12 μM. At this point in time, it required approximately 10 min to get appropriate FluoStar setting conditions.
4. 2 μL of CaCl₂ was added
5. Chymotrypsin (C4129), reconstituted in 1 mM HCl, was added at a ratio of 1:30.
6. Fluorescence development was monitored in real time every 2 minutes up to 6 hours using FluoStar (Ex/Em=540/630-12). FluoStar settings were as follows: temperature, 30° C.; 10 flashes/cycle to 180 cycles.
7. The data were plotted in an Excel graph (FIG. 11).

### B.3. Results and Discussion

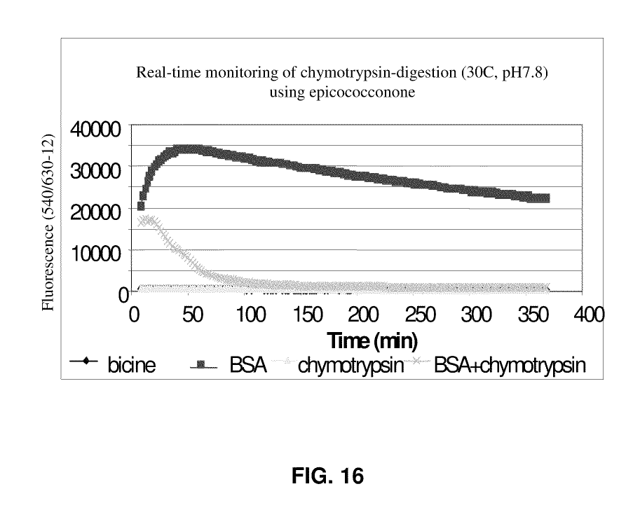

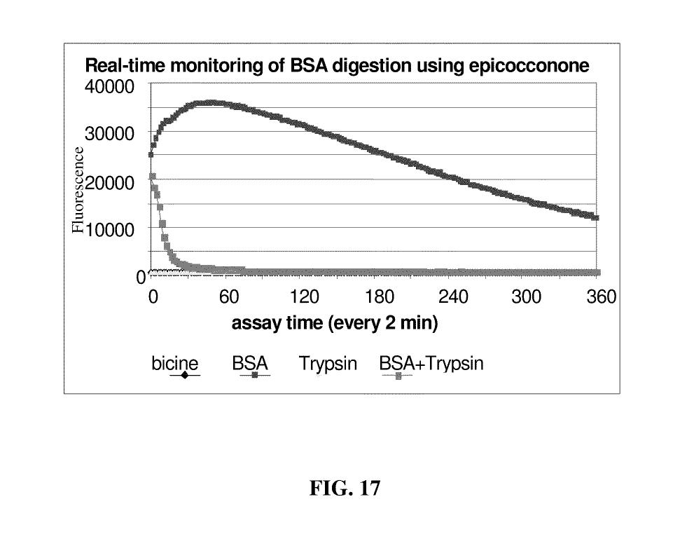

FIG. 16 shows real-time monitoring of chymotrypsin kinetics in the BSA digestion. It also displayed a similar pattern of kinetics to that of trypsin (FIG. 17) that fluorescence exponentially increased in the undigested BSA and exponentially decayed in the digested BSA. The apparent first order rate constant obtained for chymotrypsin under these conditions was 0.0447 min⁻¹.

### Example C

### Tryptic Digestion of BSA Followed by the Addition of Syproorange

### C.1 Materials

As per Example 2 above.
SYPROorange (24 mM in DMSO, Molecular Probes) diluted to 100-fold in 50 mM bicine

### C.2 Methods

As per Example 2 except the Fluorostar plate reader was set to 480 nm excitation and 600 (±10 nm) bandpass emission filter.

### C.3 Results

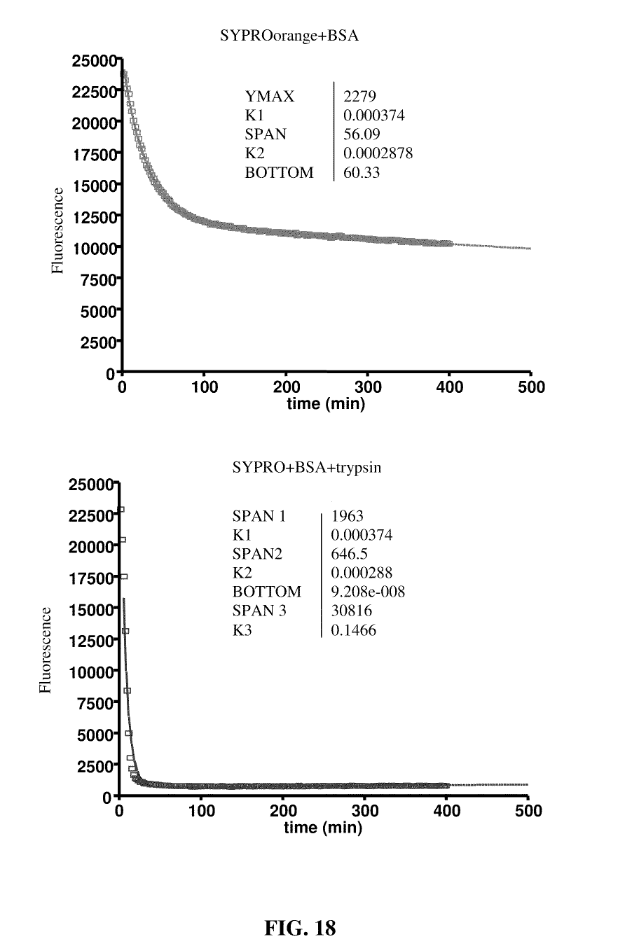

Results indicated that SYPROorange, another dye that increases fluorescence in hydrophobic environments, performed similarly to epicocconone in the real-time analysis of protein digestion (FIG. 18). In particular it was possible to extract the apparent first order rate constant for tryptic digestion by following the drop in fluorescence with time. A similar value (k=0.1466 min⁻¹) was obtained using this dye. Notable differences between epicocconone and SYPROorange are that the build-up of fluorescence observed with epicocconone is not apparent with SYPROorange and that the photobleaching/degradation of fluorescence is faster than with epicocconone.

### Example D

### Use of FluoroProfile for Quantifying Peptides Generated after Tryptic Digestion (18 hour)

### D.1 Materials and Equipment

Bicine (50 mM, pH 8.4, B3876)
BSA (10 mg/mL in 50 mM Bicine, A3059)
Trypsin (20 μg/20 μL 1 mM HCl, T6567)
Idoacetamide (1 M in 100 mM bicine, 16125)
DTT (200 mM in 100 mM bicine, Bio-rad_161-0611)
96-well plate with clear bottom (Greiner bio-one, 655096) BSA standard (A3059
BSA standard (Sigma-Aldrich, A3059)
Aprotinin standard (Sigma-Aldrich, A1153)
FluoroProfile (Sigam-Aldrich)
9200 Typhoon Scanner (Amersham Biosciences)

### D.2 Methods

1. BSA was denatured, reduced, alkylated and neutralized for tryptic digestion, as described previously and a brief summary provided below.
   100 μL of BSA sample (10 mg/mL)
   5 μL of 200 mM DTT made in biocine: 70° C. for 10 min
   4 μL of 1 M of IDA made in biocine: 45 min at RT
   20 μL of 200 mM DTT made in biocine: 45 min at RT
2. After reduction, alkylation, and neutralization, the BSA sample above was diluted 10-fold in 50 mM bicine buffer, (50 μL of the sample+450 μL 50 mM bicine buffer) to have samples for tryptic digestion, i.e. duplicate samples for undigested BSA sub-sampled at T=0 h and T=ON (over night), respectively, and duplicate samples for digested BSA (with 2.5 μL of 1 mM HCl added), and those for digested BSA (with 2.5 μL containing 2.5 ug of trypsin added) sub-sampled T=0 h and T=ON, respectively.
3. At the end of trypsin digestion, 1 μt of 10% TFA was added and stored at −80° C. The amount of BSA for digestion was calculated to be 749 μg/mL.

### D.2.1 Sample Preparation

For undigested BSA (duplicate) and digested BSA (duplicate), the samples were serially diluted 4-fold in 50 mM bicine to obtain 1 in 1024 dilution at the end.

BSA was freshly prepared in bicine buffer and serially diluted to obtain a dilution series ranged from 61 ng/mL to 1 mg/mL. Aprotinin was freshly prepared in bicine buffer and serially diluted to obtain a dilution series ranged from 61 ng/mL to 1 mg/mL.

### D.2.2 FluoroProfile Assay

A working FluoroProfile kit mix prepared from 8 parts of 50 mM bicine, 1 part of Part A, and 1 part of Part B.

Samples (50 μL) were placed into a 96-well plate and FluoroProfile working solution (50 μL) was added to each well.

Samples were incubated for 30 min at room temperature. The fluorescence was read using Typhoon scanner at 532/610 (Ex/Em).

### D.3 Results

### D.3.1 FluoroProfile Analysis of Fluorescence Levels of Undigested and Digested BSA

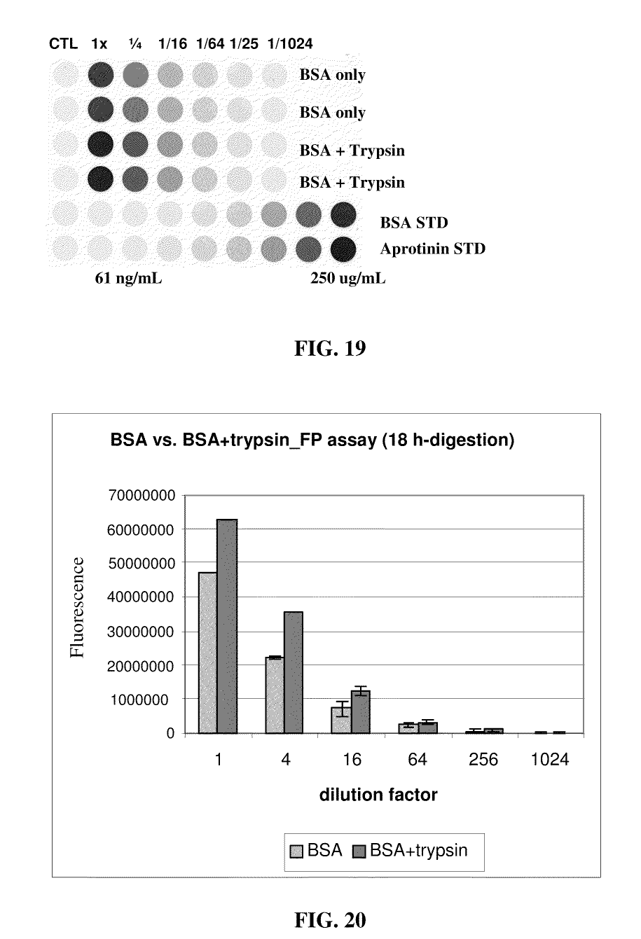

FIG. 19 shows typhoon-scanned image of the plate where the samples were assayed by FluoroProfile. As shown in Table A1 and FIG. 20, the fluorescence for digested BSA samples was significantly higher than undigested samples.

TABLE A1
Fluorosecence of undigested (3^{rd} column) and digested BSA samples (7^{th}
column) that were serially diluted. The samples were assayed by FluoroProfile
and read for fluorescence by Typhoon scanner.

| | | | | BSA + t | | | |
|---|---|---|---|---|---|---|---|
| BSA | | | | typsin | | | |
| (18 h) | average | net | | (18 h) | average | net | |
| CTL | 493601.6 | 0 | Stdev | CTL | 471418.1 | 0 | Stdev |
| (bicine) | | | | (bicine) | | | |
| 1 | 47775445 | 47281843 | 532028.6 | 1 | 63459501 | 62988083 | 12751.78 |
| ¼ | 22790727 | 22297125 | 2346631 | ¼ | 36243408 | 35771990 | 1408240 |
| 1/16 | 7727012 | 7233410 | 941509.2 | 1/16 | 12828068 | 12356650 | 808548.6 |
| 1/64 | 2750820 | 2257219 | 268100.9 | 1/64 | 3657093 | 3185675 | 218392.9 |
| 1/256 | 1235006 | 741404.5 | 109485.5 | 1/256 | 1248152 | 776734.2 | 37772.8 |
| 1/1024 | 707728 | 214126.4 | 9340.499 | 1/1024 | 696683.4 | 225265.3 | 23787.69 |

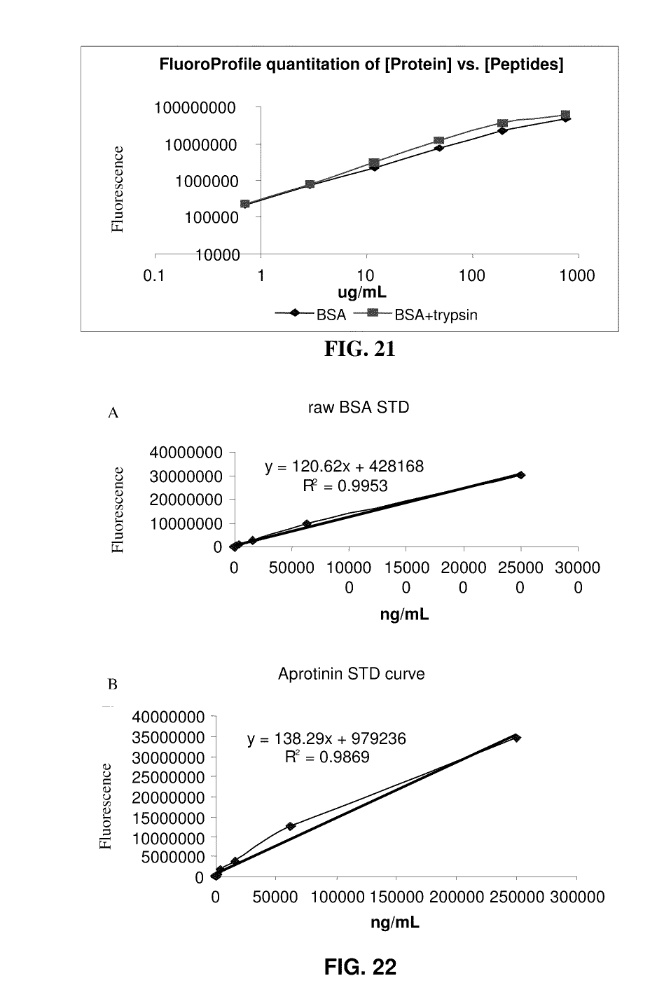

The fluorescence of the undigested and digested samples (that were serially diluted) was plotted against the BSA concentration used for tryptic digestion (Table A2 and FIG. 21). The concentration of BSA (denatured) that was used for tryptic digestion was calculated to be 749 μg/mL (see table 2).

TABLE A2
Dilution of BSA (749 μg/mL) used for tryptic digestion and
corresponding fluorescence of undigested and digested BSA

| Dilution F | μg/mL | BSA | BSA + trypsin |
|---|---|---|---|
| 1024 | 0.7 | 214126.42 | 225265.335 |
| 256 | 2.9 | 741404.525 | 776734.24 |
| 64 | 11.7 | 2257218.915 | 3185675.11 |
| 16 | 47.8 | 7233410.155 | 12356649.59 |
| 4 | 187 | 22297124.95 | 35771990.32 |
| 1 | 749 | 47281843.18 | 62988083.24 |

D.3.2 Quantitation of Digested BSA using FluoroProfile

A concentration of 704 μg/mL was determined (Table A3) in the undigested BSA (denatured, incubated for 18 hrs) by interpolation against the raw BSA standard curve (FIG. 22A). The digested BSA was estimated to be 1057 μg/mL.

TABLE A3
Quantitation of digested BSA (BSA + trypsin) using
raw BSA standard.

| | | | | Net (BSA + | |
|---|---|---|---|---|---|
| | Net BSA | average | theoretical | trypsin) | average |
| dilution F | (18 h) | (ug/mL) | value (ug/mL) | (18 h) | (ug/mL) |
| 1 | 47235629 | 704 | 749 ug/mL | 62919685 | 1057 |
| 4 | 22250910 | | | 35703592 | |
| 16 | 7187196 | | | 12288252 | |
| 64 | 2211004 | | | 3117277 | |
| 256 | 695190 | | | 708336 | |
| 1024 | 167912 | | | 156867 | |

A concentration of 560 μg/mL was determined (Table A4) in the undigested BSA (denatured, incubated for 18 hrs) by interpolation against aprotinin standard curve (FIG. 22B). The digested BSA was estimated to be 938 μg/mL.

TABLE A4
Quantitation of digested BSA (BSA + trypsin) using
aprotinin standard.

| | | | | Net (BSA + | |
|---|---|---|---|---|---|
| | Net BSA | average | theoretical | trypsin) | average |
| dilution F | (18 h) | (ug/mL) | value (ug/mL) | (18 h) | (ug/mL) |
| 1 | 47235629 | 560 | 749 ug/mL | 62919685 | 938 |
| 4 | 2225091 | | | 35703592 | |
| 16 | 7187196 | | | 12288252 | |
| 64 | 2211004 | | | 3117277 | |
| 256 | 695190 | | | 708336 | |
| 1024 | 167912 | | | 156867 | |

Fluorescence increase in the digested BSA was observed in the FluoroProfile assay where both Part A and B were used for a sub-sample taken after 18-hr tryptic digestion. Using a raw BSA standard (FIG. 22A) and aprotinin standard (FIG. 22B), the fluorescence increase of the digested BSA was observed to be approx. 50% and 67% higher than that of the undigested BSA (Table A3 and Table A4).

### Example E

### Tryptic Digestion of BSA Followed by the Addition of Nile Red

### E.1 Materials

As per Example 2 above.
Nile Red (20 mM in ethanol, Sigma-Aldrich, 73189) diluted to 100-fold in 50 mM bicine

### E.2 Methods

As per Example 2 except the Fluorostar plate reader was set to 520 nm excitation and 630 (±10 nm) bandpass emission filter.

### E.3 Results

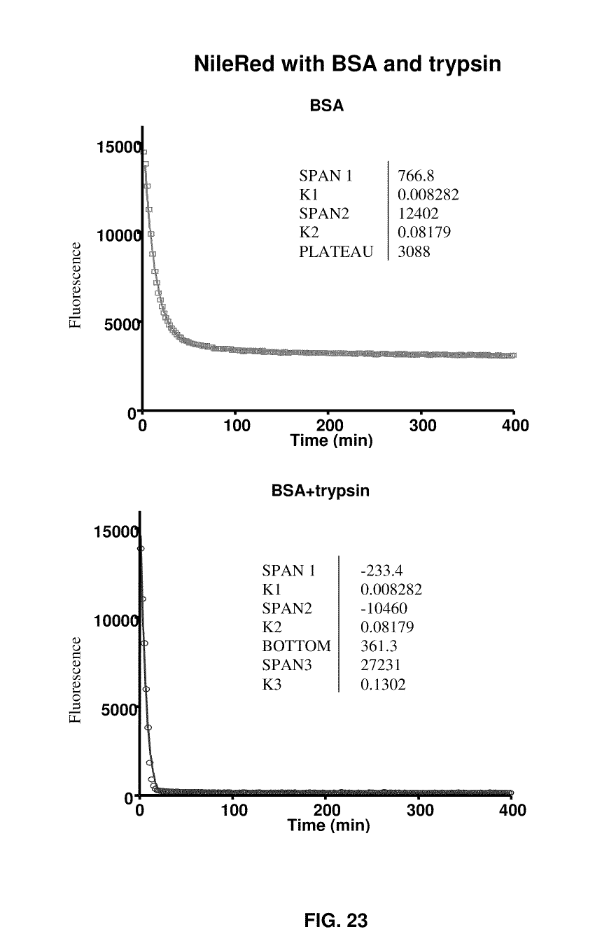

Results indicated that Nile Red also performed similarly to epicocconone and SYPROorange in the real-time analysis of protein digestion (FIG. 23). In particular it was possible to extract the apparent first order rate constant for tryptic digestion by following the drop in fluorescence with time. A similar value (k=0.1302 min⁻¹) was obtained using this dye. Notable differences between epicocconone and Nile Red are that the build-up of fluorescence observed with epicocconone is not apparent with Nile Red and that the photobleaching/degradation of fluorescence is faster than with epicocconone and faster than with SYPROorange.

In examples 1-3 and 5 it will be noted that the apparent first order kinetic constant for BSA tryptic digestion is the same (within error) for all examples shown.

### EXAMPLES

### Example 1

### Real-Time Monitoring of Glycosylase Activity Using a Fluorophore

### 1.1 Materials

Bicine (100 mM, pH 8, Sigma-Aldrich B3876)
Fetuin (20 mg/mL in RO water, Sigma-Aldrich F3004)
Peptide-N-glycosidase F (PNase F) (Sigma-Aldrich P7367)
Epicocconone (24 mM in DMSO, FLUOROtechnics)
SDS (BDH 442444H)
2-mercaptoethanol (Sigma-Aldrich M7154)
Black 96-well plates (Greiner bio-one, 655209)
Deep Purple total protein gel stain (GE Healthcare)
NuPAGE Novex 12% Bis-Tris Gels (Invitrogen, NP0341)
NuPAGE LDS sample buffer (4×, Invitrogen, NP0007)
LMW Marker (Amersham Biosciences, 17-0446-01)

### 1.2 Equipment

Typhoon 9200 (Amersham Biosciences)
FluoStar (BMG)
Electrophoresis system (XCell SureLock, Invitrogen)

### 1.3 Methods

### 1.3.1 Real-Time Monitoring of Deglycosylation Using Epicocconone in the Presence of a Detergent

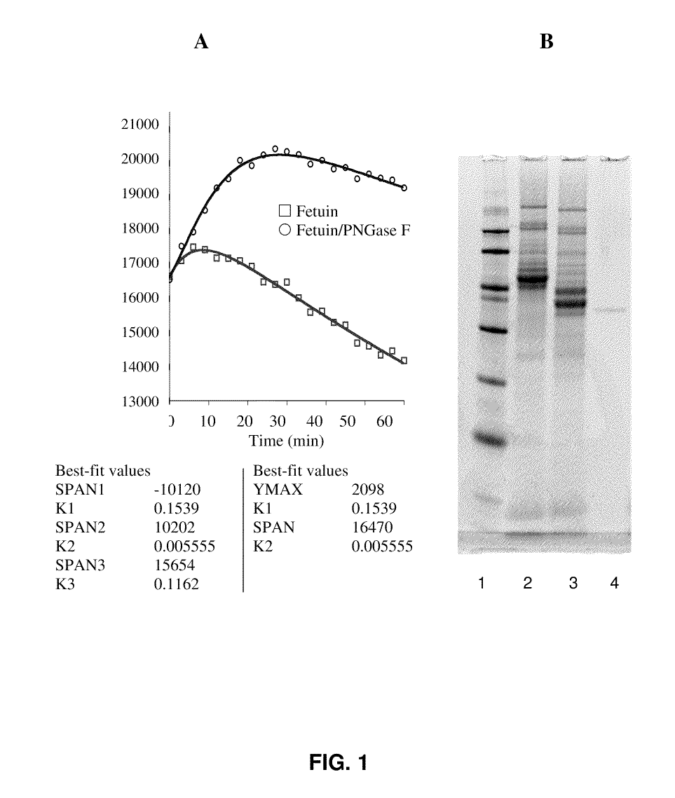

1. Fetuin protein was diluted 1:20 in the bicine buffer.
2. The protein (90 μg/90 μL) was denatured by 10 μL of a detergent (0.2% SDS with 100 mM 2-mercaptoethanol) at 100° C. for 10 minutes.
3. One hundred microlitres of the sample (step 2) was added to a microtitre plate well. Controls included a bicine-based digestion buffer, a PNGase F sample only and an unglycosylated fetuin sample (no PNGase F).
4. Epicocconone stock solution was diluted 100-fold in 100 mM bicine. One hundred microlitres of diluted epicocconone solution was added to each well.
5. The samples were allowed to equilibrate (pre-incubate) at 37° C. for 20 minutes. The FluoStar required approximately 1 min to obtain appropriate gain setting.
6. One unit of PNGase F, reconstituted in Reverse Osmosis (RO) water, was added to each futuin protein sample, and to the controls, e.g. bicine buffer+PNGase F.
7. Fluorescence development was monitored in real time every 3 minute up to 69 minutes using FluoStar (Ex/Em=540±10/630±10 nm). FluoStar settings were as follows: temperature, 37° C.; 10 flashes/cycle.
8. Progress curves were manipulated in Microsoft Excel by subtracting controls. Buffer/dye was subtracted from the sample containing fetuin/dye only and PNGase+buffer/dye was subtracted from the fetuin/PNGase/dye samples.
9. The normalised data was plotted and fitted using Prism (GraphPad v.4)—see FIG. 1. The fetuin sample was fitted to a two phase association/dissociation exponential (Y═Yₘₐₓ*(1−exp(−k₁*X))+SPAN*(exp(−k₂*X))−bottom) where Y is the fluorescence data and X is time (in minutes). The values for k₁ and k₂ were used to fit a three-phase association/dissociation exponential (Y=SPAN1*(1−exp(−k₁*X))+SPAN2*(exp(−k₂*X))+SPAN3*(1−exp(−k₃*X))−bottom) where k₃ is the pseudo-first order rate constant for the deglycosylation of fetuin by PNGase.

### 1.3.2 Visualisation of Deglycosylated Sample in SDS-PAGE

1. The sub-samples were collected at the end of the assay. The sub-samples (6.5 μL) from both digests were taken, mixed in 3.5 μL of a sample loading buffer (2.5 μL of NuPAGE sample buffer and 1 μL of 500 mM DTT) and incubated at 80° C. for 10 min.
2. Each sample was then loaded onto a 12% polyacrylamide gel (NuPAGE Bis-Tris, Invitrogen) and run (200V constant) for 50 min. until the blue loading buffer dye just ran off the gel.
3. The Deep Purple-stained gels were imaged by Typhoon scanner (Ex:Em=532:560 LP; 440 PMT).

### 1.4 Data Analysis and Results

Deglycosylation of a protein results in an increase in hydrophobicity since sugars are relatively polar. In the presence of a low concentration of a detergent, such as SDS, more detergent should associate with the protein as the sugars are cleaved. Thus, fluorescent molecules that are sensitive to their environment should respond to the change in hydrophobicity to facilitate a traceless, real-time assay for enzymatic activity, in this non-limiting example; deglycosylation of fetuin.

Fetuin (48.4 kDa), is composed of 74% polypeptide, 8.3% hexose sugars, 5.5% hexosamines and 8.7% sialic acid, and is a common glycoprotein standard.

The normalised (step 8 above), real-time, fluorescence data obtained from the reaction of fetuin with PNGase F in the presence of the fluorophore epicocconone was measured and fitted to a three-component exponential association-dissociation kinetic model using Prism (Version 4.0.3, GraphPad Software, San Diego, USA). The native fetuin sample increased slightly in fluorescence and then decreased due to photobleaching/decomposition that can be modelled using a one phase exponential association followed by a slow exponential dissociation. In contrast, the sample with enzyme increased in fluorescence and was fitted to a three phase exponential to obtain the pseudo-first order rate constant for deglycosylation. The analysis results are shown in FIG. 1.

FIG. 1A demonstrates that fluorescence increases in the sample (fetuin+PNGase F) due to the increase of hydrophobicity during deglycosylation for 1 hour at 37° C. Fitting the real-time data allows the analysis of enzyme activity on a real substrate and the determination of the kinetic constants and the half-life of hydrolysis. Ten times the half-life can be used as a measure of complete hydrolysis (59 minutes in this case). FIG. 1B is an independent SDS-PAGE validation of the real-time assay, showing the molecular shift between the native (lane 2) and PNGase F-treated (lane 3) fetuin upon deglycosylation.

### Example 2

### Real-Time Monitoring of Oligonucleotide Hydrolysis Using Three Different Fluorophores

### 2.1 Materials and Equipment

as per previous example with the following additions and/or substitutions.
Bicine (500 mM, pH 7.5, Sigma-Aldrich B3876)
MgCl₂ (1 M, Sigma-Aldrich M-8266)
CaCl₂ (1 M, BDH 010070-0500)
DNase 1 buffer (×20) containing 0.1 M bicine, 0.4 M MgCl₂ and 0.02 M CaCl₂.
Salmon sperm DNA (1.25 mg/mL in 1 RO water, Sigma-Aldrich F3004)
DNase 1 (5 mg/mL in 0.15 M NaCl, Sigma-Aldrich DN25)
Hoechst 33342 (10 mM in DMSO, Invitrogen H1399)
Propidium iodide (1.5 mM in RO water, Sigma-Aldrich P4170)
SYTOX-green (5 mM in DMSO, Invitrogen S7020)
SPP1/EcoRI DNA molecular weight marker (Breastec Ltd. DMW-S1)
DNA grade agarose (1.5% w/v, Progen 200-0011)
Nucleic acid sample loading buffer (5×, Bio-rad 161-0767)
Tris-borate-EDTA DNA running buffer (10×, Fermentas B52)
Ethidium bromide (10 mg/mL RO water, Sigma-Aldrich E7637)
Mini-Sub® Cell GT (Bio-rad)
ChemiImager™ 4400 (Alpha Innotech)

### 2.2 Methods

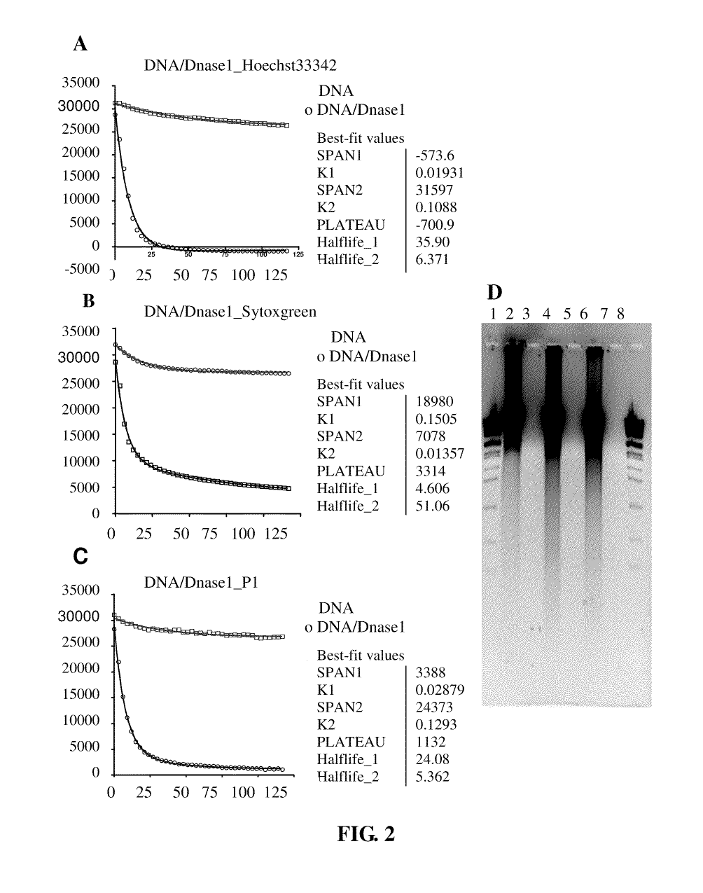

2.2.1 Real-Time Monitoring of Ds-DNA Hydrolysis with a DNase Enzyme Using Hoechst 33342, SYTOX Green and Propidium Iodide
1. DNA sample was diluted 5-fold in 1×DNase 1 buffer to give 250 μg/mL.
2. One hundred microlitres of the sample (step 1) was added to a microtitre plate. Controls included 1×DNase 1 buffer, DNase 1 sample only and an undigested DNA sample (no DNase 1).
3. Each fluorophore stock solution was diluted 100-fold in 1×DNase 1 buffer. One hundred microlitre of the diluted fluorophore solution was added to each well. The samples were equilibrated at 37° C. for 50 minutes. The FluoStar required approximately 1 min to obtain appropriate gain setting.
4. One unit of DNase 1, reconstituted in 0.15 M NaCl, was added to each DNA sample to be digested, and to one control, e.g. 1×DNase 1 buffer+DNase 1.
5. Fluorescence development was monitored in real time every 3 minute up to 120 minutes using FluoStar (Ex/Em=355/460 nm for Hoechst 33342, 540±10/630±10 for propidium iodide; 485/520 nm for SYTOX-green). FluoStar settings were as follows: temperature, 37° C.; 10 flashes/cycle.
6. Progress curves were manipulated in Microsoft Excel by subtracting controls. DNase buffer was subtracted from the sample containing DNA only and DNase+buffer was subtracted from the DNA/DNAse sample.
7. The normalised data was plotted using Prism (GraphPad v.4)—see FIG. 2. The progress curve of DNA with fluorophore is first fitted to a single phase exponential decay (Y=span*exp(−kX)+plateau) and the value for k obtained used as the fixed value for k1 in a two phase exponential decay (Y=span1*exp(−k1X)+span2*exp(−k2X)+plateau) for the DNA plus DNase enzyme to derive k2.

### 2.2.2 Visualisation of Exonuclease Activity by Agarose Electrophoresis

1. The sub-samples were collected at the end of the assay. The sub-samples (8 μL) from both digests were taken, mixed with 2 μL of a nucleic acid sample-loading buffer.
2. Each sample was then loaded onto a 2% agarose gel containing 5 μg of ethidium bromide and run (150V constant) for 1 hr until the blue loading buffer dye just ran off the gel.
   3. The ethidium bromide stained-gel was imaged by ChemiImager™ 4400.

### 2.3 Data Analysis and Results

The experiment was carried to demonstrate that fluorophores that are sensitive to their environment can be used to tracelessly follow the hydrolysis of an oligonucleotide sample, in this non-limiting example double-stranded salmon-sperm DNA with the enzyme DNase 1.

FIG. 2 shows the real-time monitoring of DNase-driven hydrolysis using three different fluorphores. An exponential decay in fluorescence was observed in all cases upon addition of DNase 1, and the pseudo-first order rate constants (K2) for hydrolysis was obtained by non-linear regression analysis. Typically, the oligonucleotide in buffer with no enzyme progress curve was fitted to a one-phase exponential decay to fit the observed slow reduction of fluorescence over time due to photobleaching and/or decomposition of the fluorophore. The enzyme-catalysed hydrolysis was fitted to a two phase exponential decay, taking the first order rate constant from the DNA only sample as one of the rate constants. The second rate constant (K2 in FIG. 2) is then a reasonable approximation for the pseudo-first order rate constant for hydrolysis of the DNA. The rates using the three different dyes agree very well, varying from 0.11 to 0.15 min⁻¹. In each case the half-life of DNA hydrolysis can be measured and 10 times this value would correspond to complete hydrolysis of the DNA (46-64 minutes in this case).

FIG. 2D is an independent DNA gel electrophoresis-based validation of the real-time assay, showing DNA samples are completely hydrolysed (lane 3, 5 and 7) by DNase 1, whereas DNA samples with no DNase added show a strong smear due to the presence of a complex mixture of DNA (lanes 2, 4 and 6). This example demonstrates the utility of several dyes in following the hydrolytic activity of an enzyme on a complex mixture of oligonucleotides (a genome).

### Example 3

### Real-Time Monitoring of Polysaccharide Hydrolysis in the Presence of a Non-Denaturing Amount of a Detergent and a Fluorophore

### 3.1 Materials and Equipment

as per previous examples with the following additions and/or substitutions.
Bicine (50 mM, pH 7, Sigma-Aldrich B3876)
Starch (1% in bicine buffer, Sigma-Aldrich S2630)
α-amylase (1.8 units/mg solid, Sigma-Aldrich A2771)
Triton X-100 (BDH 30632)

### 3.2 Methods

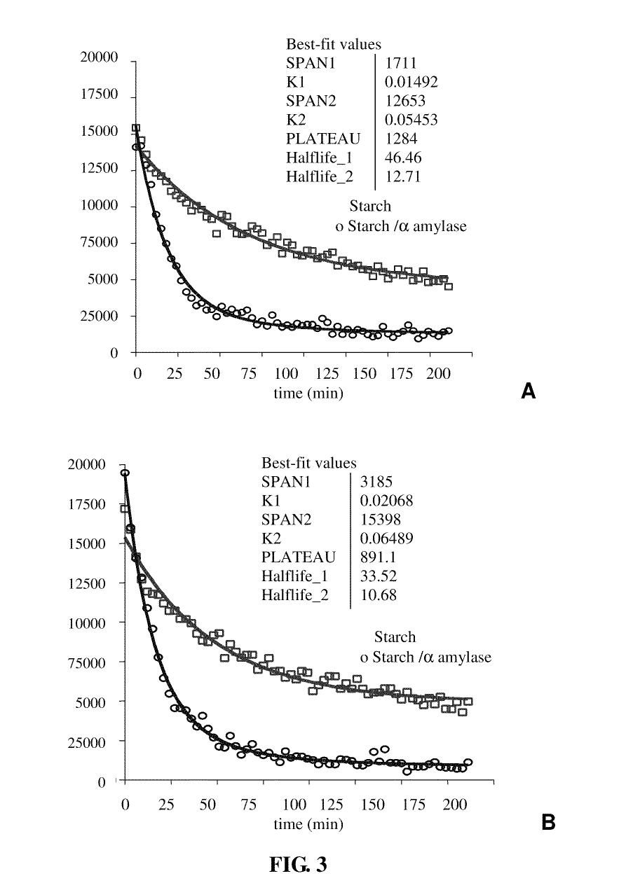

3.2.1 Real-Time Monitoring of α-Amylase-Driven Hydrolysis of Starch Monitored with Epicocconone
1. Starch solution was prepared at a concentration of 1% in 50 mM bicine buffer (pH 7) by boiling the sample for 15 minutes.
2. Triton X-100 was added to the starch solution at a final concentration of 0.02%.
3. One hundred microlitres of the sample (step 1) was added to a microtitre plate. Controls included a bicine-based digestion buffer, a α-amylase sample only and a native starch sample (no amylase).
   4. Epicocconone stock solution was diluted 100-fold in 50 mM bicine. One hundred microlitre of diluted epicocconone solution was added to each well. The samples were incubated at 37° C. for 50 minutes. The FluoStar required approximately 1 min to obtain appropriate setting conditions.
   5. Two microliter (0.036 units) of α-amylase, reconstituted in the bicine buffer, was added to one starch sample, and to one control, e.g. bicine buffer+α-amylase.
   6. Fluorescence development was monitored in real time every 3 minute up to approx. 200 minutes using FluoStar (Ex/Em=540±10/630±10 nm). FluoStar settings were as follows: temperature, 37° C.; 10 flashes/cycle.
   7. Progress curves were manipulated in Microsoft Excel by subtracting controls. Buffer was subtracted from the sample containing starch only and amylase+buffer was subtracted from the starch/amylase sample.
   8. The normalised data was plotted using Prism (GraphPad v.4)—see FIG. 3.

### 3.3 Data Analysis and Results

The experiment was carried out to demonstrate that environmentally sensitive fluorophores, such as epicocconone, can be used to monitor the hydrolysis of a carbohydrate sample, e.g. in this non-limiting example, potato starch by amylase by measuring the local hydrophobicity around the substrate.

FIG. 3 shows the real-time monitoring of amylase-driven hydrolysis using epicocconone. In the presence of the detergent triton X-100, the fluorescence signal at the beginning of the hydrolysis was ˜20% higher than without the detergent showing that the detergent binds to the starch yielding a more hydrophobic environment around the starch which is destroyed by hydrolysis leading to an exponential decrease in fluorescence. This unexpected phenomenon can be used to tracelessly follow the progress of enzymic reaction. The pseudo-first order rate constant for hydrolysis of starch by amylase was obtained by fitting a single phase exponential decay to the starch/buffer/epicocconone control and then using this value (k1) to fit a two-phase exponential decay to the starch/amylase/epicocconone sample where the first exponential is fixed at the k1 value determined for the control. The k2 value is then the pseudo-first order rate constant for the hydrolysis of starch by α-amylase. In this case the value is 0.55 min⁻¹ and 0.65 min⁻¹ in the presence of triton X-100. Complete digestion can be determined as ten times the half-life, in this case 127 and 107 minutes respectively.

### Example 4

### Real-Time Monitoring of Protein Dephosphorylation Using Hydrophobicly Active Fluorophores

### 4.1 Materials and Equipment

as per previous examples with the following additions and/or substitutions.
β-casein (Sigma-Aldrich C6905)
Alkaline phosphatase (10-30 DEA units/mg solid, Sigma-Aldrich P7640) dissolved in the bicine buffer at a concentration of 2 mg/mL
BODIPY FL C₅-ceramide (10 mM in DMSO, Invitrogen D3521)

### 4.2 Methods

### 4.2.1 Real-Time Monitoring of Phosphatase Activity Using BODIPY FL C₅-Ceramide

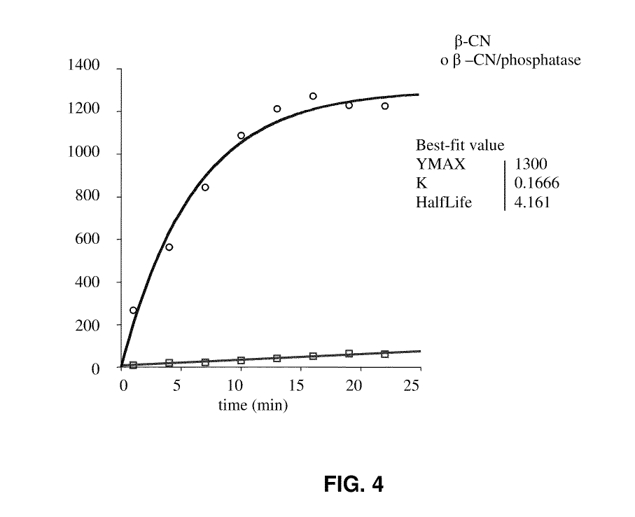

1. β-casein (β-CN) was prepared at a concentration of 1 mg/mL in 50 mM bicine buffer (pH 7.5).
2. One hundred microlitres of the sample (step 1) was added to a microtitre plate. Controls included a bicine-based digestion buffer, an alkaline phosphatase sample only and a native β-CN sample (no phosphatase).
3. BODIPY FL C₅-ceramide stock solution was diluted 100-fold in 50 mM bicine. One hundred microlitre of diluted fluorophore solution was added to each well. The samples were incubated at 30° C. for 50 minutes. The FluoStar required approximately 1 min to obtain appropriate setting conditions.
4. Two microliters of the phosphatase, reconstituted in the bicine buffer, was added to one β-CN sample, and to one control, e.g. bicine buffer+phosphatase.
5. Fluorescence development was monitored in real time every 3 minute up to approx. 30 minutes using FluoStar (Ex/Em=485/520 nm). FluoStar settings were as follows: temperature, 30° C.; 10 flashes/cycle.
6. Progress curves were manipulated in Microsoft Excel by subtracting controls. Buffer/dye was subtracted from the sample containing β-CN/dye only and phosphatase+buffer/dye was subtracted from the β-CN/phosphatase/dye samples
7. The data was plotted using Prism (GraphPad v.4)—see FIG. 4.

### 4.3 Data Analysis and Results

In this non-limiting example we show how the fluorophore BODIPY FL Cₛ-ceramide can be used to monitor alkaline phosphatase-driven hydrolysis of β-casein (β-CN). FIG. 4 shows the real-time monitoring of the dephosphorylation of a phosphoprotein by the increase in hydrophobicity associated with the removal of polar phosphate groups. The normalised data obtained from the association of BODIPY FL C₅-ceramide with casein was measured and fitted to simple first order exponential association model using Prism (Version 4.0.3, GraphPad Software, San Diego, USA). The rate constant obtained (k1) was used as a constant in a two-phase exponential increase of casein with alkaline phosphatase to obtain the pseudo-first order rate constant for the dephosphorylation reaction. The increase in fluorescence results from the increase in hydrophobicity as the phosphate groups are removed (FIG. 4). Complete hydrolysis can be calculated as 42 minutes (10×t_{1/2}) in this case.

### Example 5

### Real-Time Monitoring of Esterase Activity with Fluorophores

### 5.1 Materials and Equipment

as per previous examples with the following additions and/or substitutions.
Olive oil (no name brand, Woolworths, Australia)
Lipase (50 KLU/g, Novozymes Greasex® lipase
5-octadecanoylaminofluorescein (10 mM in DMSO, Sigma-Aldrich 74735)

### 5.2 Methods

### 5.2.1 Real-Time Monitoring of Lipase-Catalysed Hydrolysis of Olive Oil

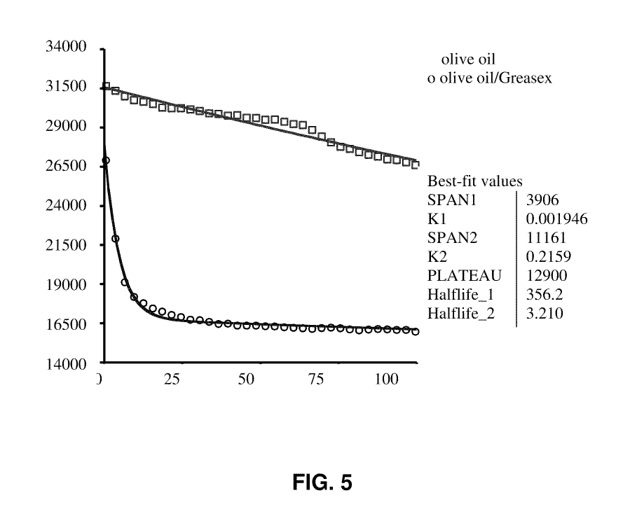

1. Olive oil was prepared in 100 mM bicine buffer at a concentration of 5%. The water/oil suspension was emulsified using Branson digital Sonifier (2×15 seconds at 60% power).
2. One hundred microlitres of the sample (step 1) was added to a microtitre plate. Controls included a bicine-based digestion buffer, lipase sample only and a native olive oil sample (no lipase).
3. 5-Octadecanoylaminofluorescein stock solution was diluted 100-fold in 100 mM bicine. One hundred microlitre of diluted fluorophore solution was added to each well.
4. The samples were incubated at room temperature for 50 minutes. The FluoStar required approximately 30 seconds to obtain appropriate gain settings.
5. Various amounts of Greasex® (lipase) tested were 0.01, 0.1, 1 and 10 μL were added to the oil samples.
6. Fluorescence development was monitored in real time every 3 minute up to approx. 200 minutes using FluoStar (Ex/Em=485/520 nm). FluoStar settings were as follows: temperature, 37° C.; 10 flashes/cycle.
7. Progress curves were manipulated in Microsoft Excel by subtracting controls. Buffer/dye was subtracted from the sample containing olive oil/dye only and Greasex+buffer/dye was subtracted from the olive oil/Greasex/dye samples
8. The normalised data was plotted using Prism (GraphPad v.4)—see FIG. 5.

### 5.3 Data Analysis and Results

In this non-limiting example, we show how a fluorophore that is sensitive to its environment, such as 5-octadecanoylaminofluorescein can be used to tracelessly follow the real-time hydrolysis of esters, such as lipids. In this case, we show the hydrolysis of olive oil by a commercial lipase but one skilled in the art would realise that the same or similar assay could be used to follow the hydrolysis of other esters with other esterases. FIG. 5 shows the real-time monitoring of hydrolysis of olive oil by the lipase Greasex® at 0.01 μL per well using 5-octadecanoylaminofluorescein as reporter. The program Prism was able to fit the progress curves to either a single phase (olive oil+buffer+dye) or two phase (olive oil+buffer+lipase+dye) exponential decay and determine the pseudo-first order rate constant for hydrolysis (FIG. 5, inset).

The exponential decrease in fluorescence observed can be rationalised as a loss of hydrophobicity upon hydrolysis of the olive oil sample into fatty acid and alcohol, which are more polar than the original ester. In this example, complete hydrolysis can be estimated as 10× the half-life (32 minutes) demonstrating the utility of this invention to the food industry.

### Example 6

### Real-Time Monitoring of Proteolysis Using Epicocconone in the Presence of a Non-Denaturing Amount of a Detergent

The experiment was carried out to investigate the real-time monitoring of protein digestion with a non-specific protease, e.g. papain using epicocconone.

### 6.1 Materials and Equipment

as per previous examples with the following additions and/or substitutions.
Protein samples: BSA (10 mg/mL in 100 mM Bicine, Sigma-Aldrich A3059), apo-transferrin (10 mg/mL in 100 mM Bicine, Sigma-Aldrich T2036), α-casein (10 mg/mL in 100 mM Bicine, Sigma-Aldrich C6780) and carbonic anhydrase (10 mg/mL in 100 mM Bicine, Sigma-Aldrich C7025).
SDS (BDH 442444H)
Papain (2 mg/mL in RO water Sigma-Aldrich P4762)
Iodoacetamide (1 M in 100 mM bicine, Sigma-Aldrich I6125)
DTT (200 mM in 100 mM bicine, BioRad 161-0611)

### 6.2 Methods

6.2.1 Hydrolysis of Different Proteins with Papain Using Epicocconone

### 6.2.1.1 Preparation of BSA for Digestion

1 Papain digestion was carried out in bicine buffer (pH 7.0)
2 Protein samples were prepared in 10 mg/mL in 100 mM bicine buffer.
3 One hundred microliter of the protein sample was used for papain digestion.

### 6.2.1.2 Reduction and Alkylation

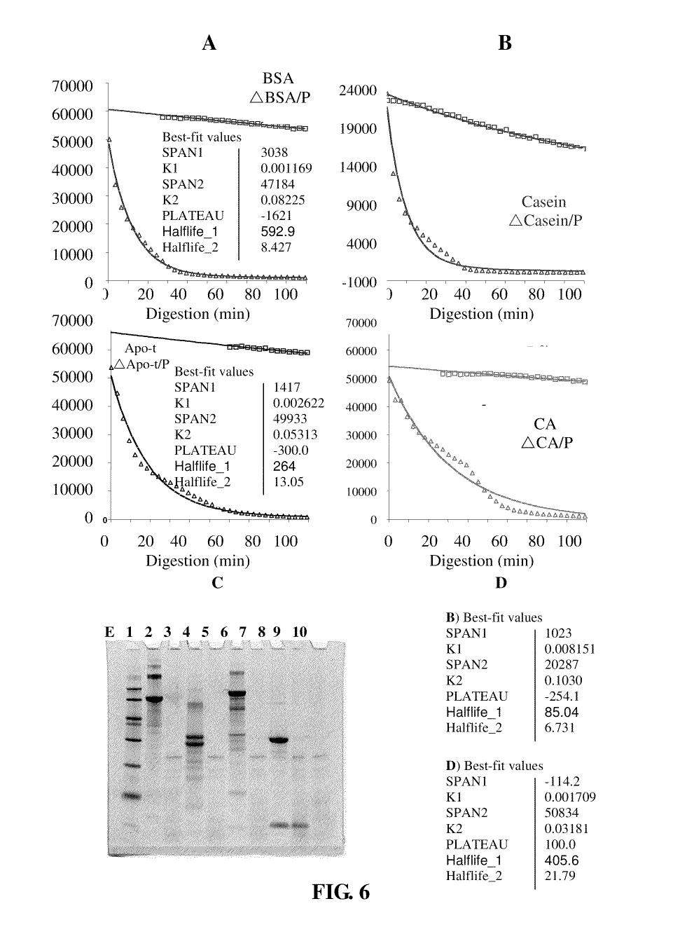

1 The 100 μL of protein samples was reduced by adding 1 μL of 10% SDS and 5 μL of DTT stock for 10 min at 80° C.
2 The samples were alkylated by adding 4 μL of the iodoacetamide stock at room temperature for 45 min-1 hr.
3 The remaining iodoacetamide of the samples were neutralised by adding 20 μL of the DTT at room temp for 45 min-1 hr.
   6.2.2 Real-Time Monitoring of Papain Digestion with Fluorophores in the Presence of a Detergent
1. The reduced and denatured protein samples were diluted 10-fold in 100 mM bicine buffer (25 μL+225 μL bicine buffer).
2. One hundred microliter of the BSA sample (step 1) was added to a well in a 96-well microtiter plate. Controls included a bicine-based digestion buffer, a papain sample only and an undigested sample (no papain). The assay consists of a 4-well sample set for BSA proteolysis using epicocconone. Three sets of samples were prepared in the same manners for the remaining protein samples, e.g. apo-transferrin, α-casein and carbonic anhydrase.
3. Epicocconone were diluted 100-fold in 100 mM bicine (pH 7.0).
4. One hundred microliter of a working fluororophore solution, was added to each corresponding well. It required approximately 90 seconds obtaining appropriate FluoStar gain setting.
5. The samples were then pre-incubated for 50 minutes at 37° C.
6. The protein samples were digested with 4 μL of papain (=0.248 units/μL) in the assay.
7. Fluorescence development was monitored in real time every 2 minutes up to 400 minutes using FluoS tar (Ex/Em=540±10/630±10).
8. Progress curves were manipulated in Microsoft Excel by subtracting controls. Buffer/dye was subtracted from the sample containing protein/dye only and papain+buffer/dye was subtracted from the protein/papain/dye samples
9. The normalised data was plotted and fitted using Prism (GraphPad v.4)—see FIG. 6.

### 6.3 Data Analysis and Results

In this non-limiting example, we show epicocconone can be used to measure proteolysis with a non-specific protease, papain. Papain is a cysteine protease but this method is also applicable to serine proteases, carboxy proteases and metaloproteases. Papain has a wide specificity (unlike trypsin or chymotrypsin) completely digesting most protein samples. Here we show the hydrolysis of BSA, casein, apotransferrin and carbonic anhydrase as examples of protein with differing properties, whose hydrolysis can be followed by our invention. FIG. 6E shows samples of protein treated with papain are completely digested after 120 minutes while proteins not treated with papain show bands corresponding to the molecular weight of the respective proteins. In some cases there are also some larger peptide fragments remaining after digestion that are resistant to further hydrolysis.

FIG. 6A-D show the progress curves generated with our invention using the fluorophore epicocconone to follow the protein digestion tracelessly and in real time. In each case, the protein with fluorophore (squares) is fitted to a single phase exponential decay (Y=span*exp(−kX)+plateau) and the value for k used as the fixed value for k1 in a two phase exponential decay (Y=span1*exp(−k1X)+span2*exp(−k2X)+plateau) for the protein plus papain (triangles). From the kinetics data, it would be possible to predict the time required for complete digestion, e.g. 10× the half-life for different proteins (see inset for half-life for each protein). The exponential decrease in fluorescence observed can be rationalised as a loss of hydrophobicity upon hydrolysis of protein samples into peptide fragments, which are far less hydrophobic than its original protein.

### Example 7

### Real-Time Monitoring of Proteolysis Using Different Fluorophores in the Presence of a Non-Denaturing Amount of a Detergent

### 7.1 Materials and Equipment

as per previous examples with the following additions and/or substitutions.
BSA (10 mg/mL in 100 mM Bicine, Sigma-Aldrich A3059)
Trypsin (20 μg/20 μL 1 mM HCl, Sigma-Aldrich T6567)
Papain (2 mg/mL in RO water Sigma-Aldrich P4762)
SDS (BDH 442444H)
Iodoacetamide (1 M in 100 mM bicine, Sigma-Aldrich I6125)
DTT (200 mM in 100 mM bicine, BioRad 161-0611)
96-well plate with clear bottom (Greiner bio-one, 655096)
SYPROorange (×5000 concentrate, Invitrogen S-6650)
Nile red (1 mg/mL in ethanol, Sigma-Aldrich N-3013)
ANS (10 mM in DMSO, Sigma-Aldrich A1028)

### 7.2 Methods

7.2.1 Hydrolysis of Proteins with Trypsin and Papain Using Epicocconone, SYPROorange, Nile Red and Anilinonaphthalene Sulfonic Acid (ANS).

### 7.2.1.1 Preparation of BSA for Digestion

1 Trypsin and papain digestion was carried out in bicine buffer at pH 8.4 and pH 7.0, respectively.
2 BSA was prepared in 10 mg/mL in 100 mM bicine buffer.
3 One hundred microliter of the BSA sample was used for trypsin and papain digestion.

### 7.2.1.2 Reduction and Alkylation

BSA sample was reduced and alkylated, as described previously (section 6.2.1.2).

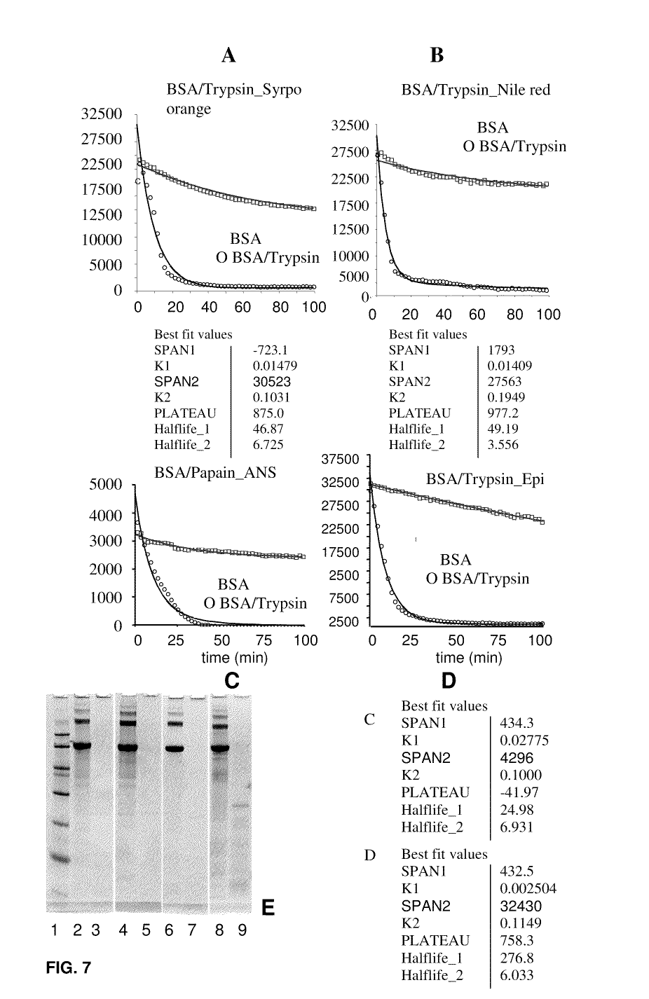

7.2.2 Real-Time Monitoring of Trypsin Digestion with Fluorophores in the Presence of a Detergent
1. The reduced and denatured BSA sample was diluted 10-fold in 100 mM bicine buffer (25 μL+225 μL bicine buffer).
2. One hundred microliter of the sample (step 1) was added to a well in a 96-well microtiter plate. Controls included a bicine-based digestion buffer, a trypsin sample or papain only and an undigested BSA sample (no trypsin or no papain). The assay consists of a 4-well sample set for one fluorophore, e.g. SYPROorange. Three sets of samples were prepared in the same manners for the remaining fluorophores, e.g. Nile red, epicocconone and SYPROorange.
3. SYPROorange stock solution was diluted 5000-fold in the bicine buffer (pH 8.4). Nile red and epicocconone and stock solutions were diluted 100-fold in the 100 mM bicine (pH 8.5). ANS was diluted 100-fold in the bicine buffer (pH 7.0).
4. One hundred microliter of a working fluororophore solution, e.g. SYPROorange, Nile red, epicocconone and ANS, was added to each corresponding well. It required approximately 30 seconds to get appropriate FluoStar setting conditions for Nile red and epicocconone, and 90 seconds for SYPROorange and ANS.
5. The samples were then pre-incubated for 50 minutes at 37° C.
   a) Trypsin (Sigma-Aldrich T6567), reconstituted in 1 mM HCl, was added to the samples to be digested at a ratio of 1:40. The same amount of the enzyme was added to a control containing only the buffer component.
   b) Four microliter of papain (2 mg/mL) was added to the samples to be digested. The same amount of the enzyme was added to a control containing only the buffer component.
6. Fluorescence development was monitored in real time every 2 minutes up to 400 minutes using FluoStar (Ex/Em=485/600±10 for SYPROorange; 540±10/630±10 for epicocconone and Nile red; 355/460 for ANS). FluoStar settings were as follows: temperature, 37° C.; 10 flashes/cycle.
7. Progress curves were manipulated in Microsoft Excel by subtracting controls. Buffer/dye was subtracted from the sample containing protein/dye only and enzyme+buffer/dye was subtracted from the protein/enzyme/dye samples. The normalised data was plotted and fitted using Prism (GraphPad v.4)—see FIG. 7

### 7.3 Data Analysis and Results

FIG. 7 shows real-time monitoring of proteolysis, e.g. BSA/trypsin and BSA/papain using four different fluorohpores. The fluorophores in the example were tested for ability to measure original status of hydrophobicity in a protein (before hydrolysis) and subsequent status of hydrophobicity in the protein (upon hydrolysis).

The normalised data were used for Prism analysis for obtaining the rate constants. In each case, the protein with fluorophore (open squares) is fitted to a single phase exponential decay (Y=span*exp(−kX)+plateau) and the value for k used as the fixed value for k1 in a two phase exponential decay (Y=span1*exp(−k1X)+span2*exp(−k2X)+plateau) for the protein plus papain (open circles). Through this method it was possible to measure the rate constants for hydrolysis and predict the time point for complete hydrolysis. FIG. 7E is an independent validation of digestion using SDS-PAGE of the actual real-time samples, showing complete digestion of BSA with trypsin or papain in the presence of different fluorophores. It can be seen from this figure that the bands associated with the protein have disappeared after 120 minutes and that only a faint band for residual papain or trypsin can be seen. In some cases there are also some larger peptide fragments remaining after digestion that are resistant to further hydrolysis.

Although the invention has been described with reference to specific examples, it will be appreciated by those skilled in the art that the invention may be embodied in many other forms.

### Example 8

### An Alternative Method to Monitor Hydrolytic Activity Using Environmentally Sensitive Fluorophores by Sub-Sampling

### 8.1 Materials and Equipment

as per previous examples with the following additions and/or substitutions
Epicocconone (1 mg/mL 20% ACN/80% DMSO; Fluorotechnics)
Protein samples, bovine serum albumin (A-3059, Lot 083K1291) and carbonic anhydrase (C-7025, Lot 093K9310)
SDS (BDH 44244411)
Trypsin (20 μg/20 μL 1 mM HCl; T6567, Sigma-Aldrich)
Leupeptin (L2884, Lot064K86283, Sigma-Aldrich)
Trypsin inhibitor (Type II-S, T9128, Lot025K7014)

### 8.2 Methods

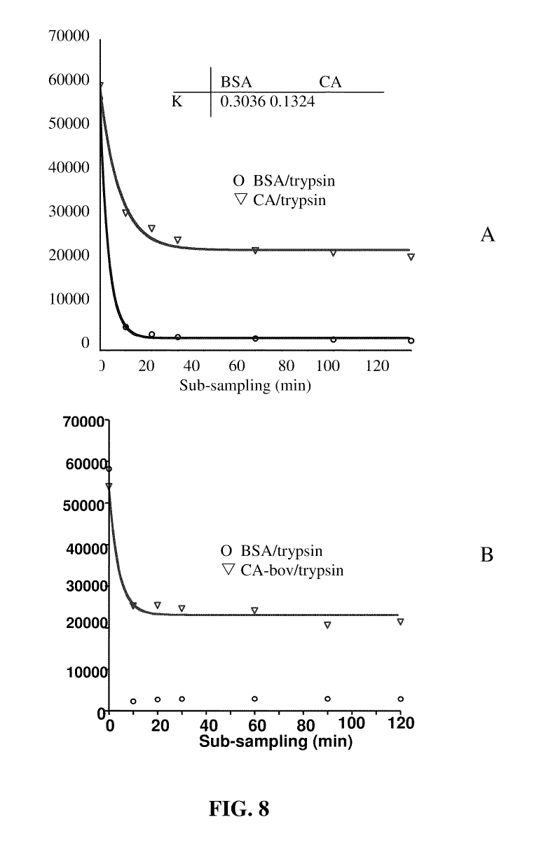

1. The trypsin inhibitors, leupeptin and trypsin-inhibitor (soybean) were prepared at a concentration of 1 mM in RO water and 0.48 mg/mL in RO water, respectively.
2. The protein samples were prepared as previously described in Example 6. The reduced and alkylated protein samples were diluted 1:10 in 100 mM bicine buffer (pH 8.4-5).
3. Trypsin (4.62 μg) was added to each protein sample (231 μg/300 μL) at a ratio of 1:50 and the samples were incubated at 37° C.
4. Sub-samples were collected for inhibition of trypsin activity either by leupeptin or by trypsin inhibitor (soybean).
   a. Forty-five microlitres of the tryptic digests were sub-sampled at 0, 10, 20, 30, 60, 60, 90, and 120 minutes and immediately added to 1.5 mL tubes containing 5 μL of 1 mM leupeptin. The sub-samples treated with leupeptin inhibitor were left at room temperature until fluorescence was read.
   b. Forty-five microliters of the tryptic digests were sub-sampled at 0, 10, 20, 30, 60, 60, 90, and 120 minutes and immediately added to 1.5 mL tubes containing 5 μL of soybean trypsin inhibitor. The sub-samples treated with soybean trypsin inhibitor were left at room temperature until fluorescence was read.
5. Controls for leupeptin inhibitor included the bicine buffer only and the bicine buffer containing trypsin or trypsin+leupeptin. Controls for trypsin inhibitor (soybean) also included the bicine buffer only, the bicine buffer containing trypsin or trypsin+soybean trypsin inhibitor.
6. Epicocconone solution was prepared by diluting it 1:100 in 100 mM bicine (pH 8.4-8.5).
7. The sub-samples (40 μL), as prepared above, were added to a 96-well plate, to which an equal volume of epicocconone solution was added. The plate was inserted into FluoStar and incubated for 50 min at 37° C.
8. Fluorescence of the sub-samples was read at Ex/Em=540-10/630-10 nm with 10 flashes.
9. The fluorescence value was normalised by subtracting basal fluorescence of corresponding controls from the raw fluorescence of the sub-samples.
10. The normalised data was plotted and can be seen in FIG. 8.

### 8.3 Data Analysis and Results

FIG. 8 shows the fluorescence decay of tryptic digestion of BSA and CA that were sub-sampled at various time points. At each sub-sample, the trypsin activity was inhibited either by leupeptin (A) or by soybean trypsin inhibitor (B). This alternative method was tested for its applicability to monitoring Bovine Serum Albumin (BSA) or Carbonic Anhydrase (CA) tryptic digestion, and produced similar fluorescence decay results compared to the results generated from the real-time assay (eg Example 6).

The rates of digestion of both BSA and CA in the present method appeared to be slightly faster than those in the real-time assay. This could be due to the time required to effect complete inhibition of trypsin by the inhibitors, or that the tryptic digestion runs slightly faster in the absence of a fluorophore.

An alternative embodiment of the invention includes the running of hydrolytic digestion without the presence of a dye. In this example, the tryptic digestion of two proteins can be followed by sub-sampling and quenching of the digestion with protease inhibitors and then adding the dye and measuring fluorescence. Considering that the inhibition of trypsin is time dependant and may not be complete, the measured pseudo-first order rate constant for digestion of BSA (0.3 min⁻¹) was similar to that found by the method of example 7 (0.1-0.2 min⁻¹).

### Example 9

### Real-Time Monitoring of Hydrolytic Activity in a Complex Proteome Using a Fluorescent Reporter Dye

### 9.1 Materials and Equipment

As per Example 7.
Compressed baker's yeast (Microbiogen Pty Ltd, Australia)
NaOH (1 M, Sigma-Aldrich 480878)
FluoroProfile (Sigma-Aldrich FP0010)

### 9.2 Methods

### 9.2.1 Yeast Preparation

1 A small pellet of compressed baker's yeast (100 mg) was used for trypsin digestion.
2 The pellet was suspended in 2 mL of 1 M NaOH. The sample was then centrifuged at 2100×g for 10 min.
3 An aliquot of the supernatant was diluted 5-fold in RO water to reduce the NaOH concentration to 200 mM.
4 The protein content was measured at 1.3 mg/mL by using Fluor Profile Kit.
5 A small aliquot of the protein extract (15 μL) was mixed with 85 μL of bicine buffer (50 mM, pH 8.5).
6 One hundred microlitres of the final yeast sample was used for trypsin digestion.

### 9.2.2 Real-Time Monitoring of Tryptic Digestion of Yeast Proteome Using Epicocconone

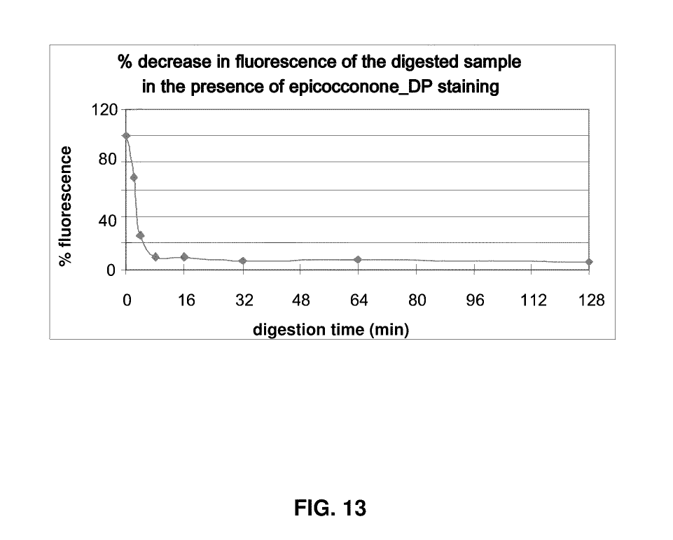

1 One hundred microlitres of the final yeast sample was added to a microtitre plate well. Controls included a bicine-based digestion buffer, a trypsin sample only and an undigested yeast sample (no trypsin).
3 Epicocconone stock solution was diluted 100-fold in 50 mM bicine. One hundred microlitres of diluted epicocconone solution was added to each well, making a 4-well assay. The FluoStar required approximately 30 seconds obtaining appropriate setting conditions.
4 Trypsin (Sigma-Aldrich T6567), reconstituted in 1 mM HCl, was added at a ratio of 1:20.
5 Fluorescence development was monitored in real time every 2 minutes up to 400 minutes using FluoStar (Ex/Em=540±10/630±10 nm). FluoStar settings were as follows: temperature, 37° C.; 10 flashes/cycle.
6 Progress curves were manipulated in Microsoft Excel by subtracting controls. Bicine buffer was subtracted from the sample containing protein only and trypsin+buffer was subtracted from the protein/trypsin sample.
7 The normalised data was plotted using Prism (GraphPad v.4)—see FIG. 13. The progress curve for the yeast proteome with epicocconone was fitted to a two phase exponential association/dissociation (Y=Ymax*(1−exp(−k1*X))+span*(exp(−k2*X))−bottom) to derive values for k1 (association constant) and k2 (dissociation constant). These values were used to fit the tryptic digestion of the yeast proteome to a three phase exponential keeping k1 and k2 fixed to the values found above into the equation Y=span1*(1−exp(−k1*X))+span2*(exp(−k2*X))+span3 exp(−k3*X)−bottom to derive a value for k3.

### 9.3 Data Analysis and Results

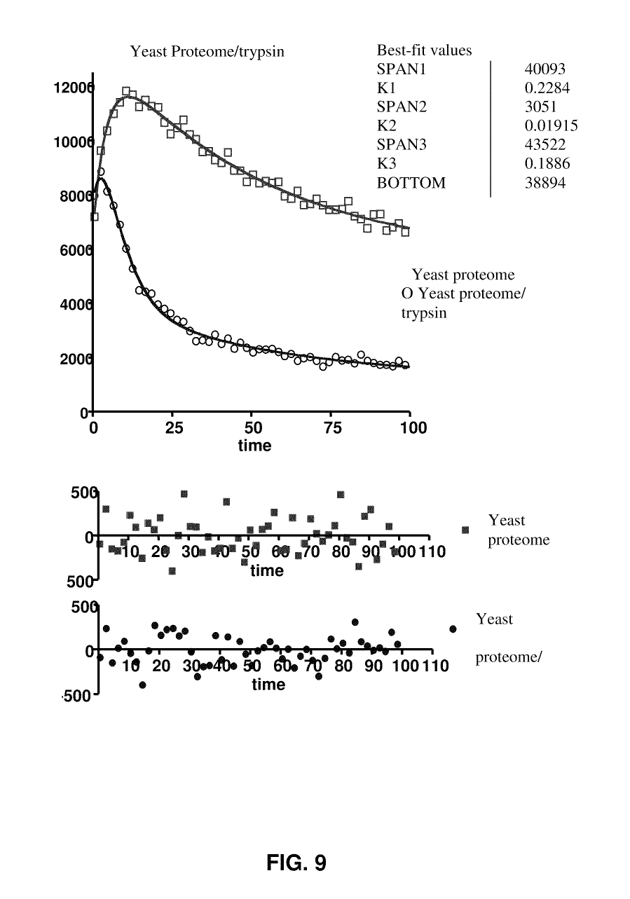

This example shows the utility of the method for monitoring the hydrolytic activity in a complex protein mixture, in the non-limiting example, a yeast proteome. FIG. 9 shows the real-time monitoring of tryptic digestion of a yeast proteome using the hydrophobicly active dye epicocconone and the detergent SDS to follow the digestion through a decrease in fluorescence as the proteins are hydrolysed. In contrast, the sample of yeast proteome with epiccconone and no trypsin results in an initial increase in fluorescence due to the time-dependant association of epicocconone with the proteins and then a slow exponential decrease due to photobleaching and/or decomposition of the fluorophore and/or fluorophore-protein adduct. This can be modelled to a two-phase exponential association/dissociation to obtain the pseudo-first order rate constants for these processes. These values can then be used to determine the pseudo-first order rate constants for hydrolysis of the complex mixture by non-linear regression. The residues from non-linear fitting of the two-phase exponential association/dissociation (solid squares) indicate that there is a good fit between experimental data and theory for the association between reporter dye and proteome. Similarly, the residual of the three-phase exponential show an equally good fit (solid circles) indicating that the determined pseudo-first order rate constant (k3) for hydrolysis of a complex proteome is accurately determined.

### Example 10

### Real-Time Monitoring of Proteolysis Using a Fluorophore with and without Detergent

This example relates to the effect of detergent on the fluorescence output of hydrophobicly active dyes in the context of real-time monitoring of hydrolysis.

### 10.1 Materials and Equipment

as per example 6

### 10.2 Methods

10.2.1 Hydrolysis of Carbonic Anhydrase (CA) Using Epicocconone with and without SDS

### 10.2.1.1 Preparation of BSA for Digestion

1 Trypsin digestion was carried out in bicine buffer (pH 8.4)
2 Protein samples were prepared in 10 mg/mL in 100 mM bicine buffer.
3 One hundred microliter of the protein sample was used for trypsin digestion.

### 10.2.1.2 Reduction and Alkylation

4. The 100 μL of protein samples was reduced by adding 1 μL of 10% SDS and 5 μL of DTT stock for 10 min at 80° C.
5. Another sample was reduced with only 5 μL of DTT stock for 10 min at 80° C. (no SDS)
3 The samples were alkylated by adding 4 μL of the iodoacetamide stock at room temperature for 45 min-1 hr.
4 The remaining iodoacetamide of the samples were neutralised by adding 20 μL of the DTT at room temp for 45 min-1 hr.
   10.2.2 Real-Time Monitoring of Trypsin Digestion with Fluorophores in the Presence or Absence of a Detergent
10. The reduced and denatured protein samples were diluted 10-fold in 100 mM bicine buffer (25 μL+225 μL bicine buffer).
11. One hundred microliter of the CA sample (step 1) was added to a well in a 96-well microtiter plate. Controls included a bicine-based digestion buffer, a papain sample only and an undigested sample (no papain). The assay consists of a 4-well sample set for CA proteolysis using epicocconone.
12. Epicocconone were diluted 100-fold in 100 mM bicine (pH 8.4).
13. One hundred microliter of a working fluororophore solution was added to each corresponding well. It required approximately 90 seconds obtaining appropriate FluoStar gain setting.
14. The protein samples were digested with 4 μL of trypsin solution in the assay.
15. Fluorescence development was monitored in real time every 2 minutes up to 400 minutes using FluoStar (Ex/Em=540±10/630±10).
16. Progress curves were manipulated in Microsoft Excel by subtracting controls. Buffer/dye was subtracted from the sample containing protein/dye only and papain+buffer/dye was subtracted from the protein/papain/dye samples
17. The normalised data was plotted and fitted using Prism (GraphPad v.4)—see FIG. 6.

### 10.3 Data Analysis and Results

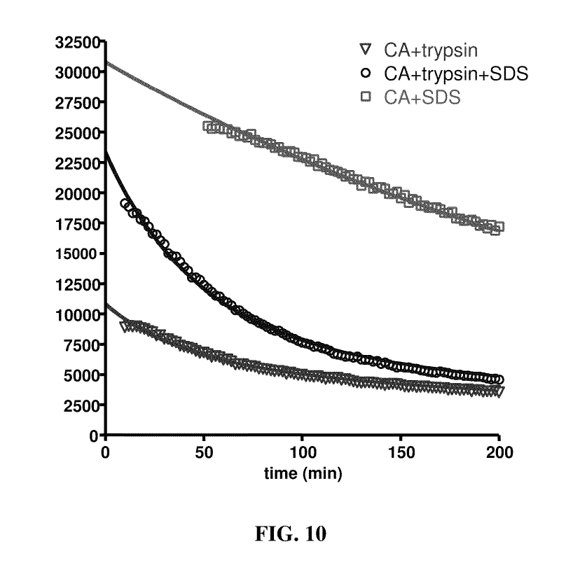

Progress curves were fitted as per Example 6. (FIG. 10). In the presence of a non-denaturing quantity of detergent (open squares) carbonic anhydrase (CA) becomes more hydrophobic and has an increase in fluorescence when exposed to a fluorophore that is sensitive to its environment such as epicocconone. Upon addition of trypsin, the fluorescence drops as per Example 6 but in the sample with no SDS (inverted triangles) the change in fluorescence is reduced by more than 50% compared to in the presence of SDS (open circles). In both cases the observed rate constant was similar (0.025 min⁻¹) but the 95% confidence interval for the sample without SDS was much higher due to the much lower signal. This example demonstrates the importance of small amounts of detergent in real-time monitoring of hydrolytic activity by hydrophobicly active fluorophores.

1. A method of measuring the activity of a hydrolytic agent comprising:
step 1: contacting a biomolecule with a hydrolytic agent in the presence of a fluorescent dye under conditions which allow digestion of the biomolecule by the hydrolytic agent; and
step 2: monitoring fluorescence of the dye over time,
wherein a change in fluorescence over time signifies digestion of the biomolecule by the hydrolytic agent.

2. A method of determining an end-point for digestion of a biomolecule by a hydrolytic agent comprising:
step 1: contacting a biomolecule with a hydrolytic agent in the presence of a fluorescent dye under conditions which allow digestion of the biomolecule by the hydrolytic agent, and
step 2: monitoring a change in fluorescence of the dye over time,
wherein the absence of a further change in fluorescence signifies the end-point for digestion of the biomolecule.

3. A method of monitoring digestion of a biomolecule by a hydrolytic agent comprising:
step 1: contacting a biomolecule with a hydrolytic agent to form a reaction mixture,
step 2: contacting a first sample of the reaction mixture with a fluorescent dye
and determining fluorescence of first sample,
step 3: subjecting the reaction mixture of step 1 to conditions which allow digestion of the biomolecule by the hydrolytic agent, and
step 4: at a desired time point during digestion of the biomolecule, contacting a second sample of the reaction mixture with a fluorescent dye; and
step 5: determining fluorescence of the second sample,
wherein a change in fluorescence of the second sample when compared to the first sample signifies the degree of digestion of the biomolecule by the hydrolytic agent.

4. A method according to claim 3 further including the steps of, where necessary:
additionally sampling the reaction mixture at intervals during digestion and, after addition of a fluorescent dye to each additional sample, determining fluorescence of the additional sample.

5. A method according to claim 4 including repeating sampling of the mixture, addition of the dye and determining the fluorescence until no further change in fluorescence is observed.

6. A method according to any one of the claims 3 to 5 wherein said samples are quenched.

7. A method for measuring and/or detecting products of a hydrolytic digestion reaction comprising:
step 1: subjecting a biomolecule to hydrolytic digestion to obtain protein or peptide fragments,
step 2: contacting said protein or peptide fragments with a fluorescent dye, and
step 3: detecting a change in fluorescence of the dye,
wherein said change in fluorescence of the dye is proportional to the quantity of said protein or peptide fragments.

8. A method according to any one of the preceding claims wherein said biomolecule is a biological macromolecule.

9. A method according to any one of the preceding claims wherein said biomolecule is hydrolysable.

10. A method according to any one of the preceding claims wherein said biomolecule is chosen from the group consisting of carbohydrates, oligonucleotides, proteins, peptides, lipids and mixtures thereof.

11. A method according to claim 10 wherein said biomolecule is present in a genome, proteome or cellular extract.

12. A method according to any one of the preceding claims wherein said hydrolytic agent changes the hydrophobicity of said biomolecule.

13. A method according to any one of the preceding claims wherein a detergent added in a non-denaturing amount.

14. A method according to claim 13 wherein said detergent is chosen from the group consisting of SDS, LDS, triton X-100, CHAPS, ALS, CTAB, DDAO and DOC.

15. A method according to claim 13 or claim 14 wherein addition of said detergent changes hydrophobicity of said biomolecule, thereby affecting binding of said fluorescent dye to said biomolecule.

16. A method according to any one of the preceding claims wherein said hydrolytic agent is an enzyme.

17. A method according to any one of claims 1-15 wherein said hydrolytic agent is a protease, esterase, glycosylase, phosphatase or nuclease capable of cleaving said biomolecule in at least one position.

18. A method according to claim 17 wherein the protease is chosen from the families consisting of aminopeptidases, dipeptidases, dipeptidyl-peptidases and tripeptidyl-peptidases, peptidyl-dipeptidases, serine-type carboxypeptidases, metallocarboxypeptidases, cysteine-type carboxypeptidases, omega peptidases, serine endopeptidases, cysteine endopeptidases, aspartic endopeptidases, metalloendopeptidases, threonine endopeptidases.

19. A method according to claim 17 wherein the esterase is chosen from the families consisting of carboxylic ester hydrolases, thiolester hydrolases, phosphoric monoester hydrolases, phosphoric diester hydrolases, triphosphoric monoester hydrolases, sulfuric ester hydrolases, diphosphoric monoester hydrolases, phosphoric triester hydrolases, exodeoxyribonucleases producing 5′-phosphomonoesters, exoribonucleases producing 5′-phosphomonoesters, exoribonucleases producing 3′-phosphomonoesters, exonucleases active with either ribo- or deoxyribonucleic acid, exonucleases active with either ribo- or deoxyribonucleic acid, endodeoxyribonucleases producing 5′-phosphomonoesters, endodeoxyribonucleases producing other than 5′-phosphomonoesters, site-specific endodeoxyribonucleases specific for altered bases, endoribonucleases producing 5′-phosphomonoesters, endoribonucleases producing other than 5′-phosphomonoesters, endoribonucleases active with either ribo- or deoxyribonucleic, endoribonucleases active with either ribo- or deoxyribonucleic acids.

20. A method according to claim 17 wherein the glycosylase is chosen from the families consisting of glycosidases (enzymes hydrolyzing N-, O- and S-glycosyl groups).

21. A method according to any one of claims 7 to 20 wherein said fluorescent dye binds or interacts with said biomolecule hydrophobicly.

22. A method according to claim 21 wherein said fluorescent dye substantially changes its fluorescent behaviour in response to the lipophilicity of its environment.

23. A method according to any one of the preceding claims wherein said fluorescent dye is selected from the group consisting of epicocconone, the cyanine dyes, laurdan/prodan family of dyes, dapoxyl derivatives, pyrene dyes, diphenylhexatriene derivatives, ANS and its analogues, styryl dyes, amphiphilic fluoresceins, rhodamines and coumarins.

24. A method according to any one of the preceding claims wherein said fluorescent dye is selected from the group consisting of epicocconone, SYTOX green, Hoechst 33342, propidium iodide, BODIPY FL C₅-ceramide, 5-octadecanoylaminofluorescein, SYPROorange or Nile red.

25. A method according to any one of the preceding claims wherein said hydrolysis is carried out in the presence of a buffer.

26. A method according to claim 25 wherein the buffer is a Good's buffer.

27. A method according to claim 25 wherein the buffer is a bicine buffer.

28. A method according to any one of the preceding claims wherein hydrolysis of said biomolecule is substantially unaffected by said fluorescent dye.

29. A method according to any one of the preceding claims wherein fluorescence is measured over time to provide data indicative of a reaction rate coefficient.

30. A method according to any one of the preceding claims wherein digestion is stopped when an end point is achieved.

31. A method according to claim 30 wherein further analysis of the reaction mixture takes place after digestion is stopped.

32. A method according to claim 31 wherein further analysis is selected from the group consisting of peptide mass finger printing (PMF), peptide mapping and HPLC.

33. A method according to any one of the preceding claims further including the addition of a base to said fluorescent dye.

34. A method according to any one of the preceding claims wherein said biomolecule is derived from a biological sample or food sample.

35. A method according to claim 34 wherein said biomolecule is a protein or mixture of proteins.

36. A method according to claim 34 wherein said biomolecule is a carbohydrate or mixture of carbohydrates.

37. A method according to claim 34 wherein said biomolecule is a glycoprotein or starch.

38. A method according to claim 34 wherein said biomolecule is a lipid.

39. A method according to claim 34 wherein said biomolecule is a vegetable oil.

40. A method according to claim 34 wherein said biomolecule is an oligonucleotide.

41. A method according to claim 34 wherein said biomolecule is DNA.

42. A kit for use in the method of any one of the previous claims comprising:
a fluorescent dye,
one or more hydrolytic agents,
optionally a standard substrate for the hydrolytic agent, and
instructions on how to use the kit for monitoring digestion of the biomolecule.

43. A kit according to claim 42 further including a standard protein or peptide substrate.

44. A kit according to claim 43 wherein said substrate is chosen from the group consisting of BSA, apo-transferrin, α-casein, β-casein, carbonic anhydrase, fetuin, salmon sperm DNA, soluble starch, and olive oil.

45. A kit according to any one of claims 42 to 44 further including a buffer.

46. A kit according to claim 45 wherein said buffer is a Good's buffer.

47. A kit according to claim 45 wherein said buffer is a bicine buffer.
